(19) United States
(12) Patent Application Publication
(10) Pub. No.: US 2010/0239590 A1
(43) Pub. Date: Sep. 23, 2010

(54) JOINT DESTRUCTION BIOMARKERS FOR ANTI-IL-17A THERAPY OF INFLAMMATORY JOINT DISEASE

(75) Inventors: Edward Paul Bowman, Redwood City, CA (US); Cheng-Chi Chao, San Jose, CA (US); Shi-Juan Chen, San Ramon, CA (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/663,381

(22) PCT Filed: Jun. 20, 2008
(86) PCT No.: PCT/US08/07770

§ 371 Date: May 24, 2010

Related U.S. Application Data
(60) Provisional application No. 60/945,239, filed on Jun. 20, 2007.

(51) Int. Cl. A61K 39/395; G01N 33/53; C07K 16/24

(52) U.S. Cl. 424/145.1; 436/501; 530/388.23

(57) ABSTRACT

Novel methods and drug products for treating inflammatory joint diseases such as rheumatoid arthritis and associated arthritides are disclosed. The methods and products employ various serum markers of bone and cartilage metabolism or destruction, including cartilage oligomer matrix protein (COMP) and Receptor activator of NFB ligand (RANKL), as biomarkers to assess the effect of IL-17A antagonists on joint destruction in inflammatory joint diseases.

The present application claims the benefit of U.S. Provisional Patent Application 60/945,239, filed 20 Jun. 2007.

### FIELD OF THE INVENTION

The present invention relates generally to the treatment of inflammatory joint diseases with antagonists of interleukin-17A (IL-17A). More specifically, the invention relates to biomarkers that are correlated with the efficacy of IL-17A antagonists for inhibiting joint destruction in rheumatoid arthritis and associated arthritides.

### BACKGROUND OF THE INVENTION

Rheumatoid arthritis (RA) is an inflammatory disease caused by the dys-regulation of the immune system resulting in joint inflammation, causing joint pain, discomfort, swelling and stiffness, with progressive bone and cartilage erosion. The combination of inflammation and structural joint damage results in loss of function which can lead to permanent disability.

IL-17A, which was originally named cytotoxic T-Lymphocyte-associated antigen 8 (CTLA8) is a homodimeric cytokine that binds to IL-17RA (also known as IL17R) and IL-17RC. The functional receptor for IL-17A is believed to be a multimeric receptor complex comprising one or both of IL-17RA and IL-17RC (e.g., an IL-17RA homodimer, an IL-17RC homodimer, or an IL-17RA/IL-17RC heterodimer) and possibly a third, as yet unknown, protein (Toy, D. et al., (2006) J. of Immunol. 177(1):36-39; unpublished data).

IL-17A is produced by a subset of T cells known as Th17 cells, whose differentiation is initiated by TGF-beta signaling in the context of proinflammatory cytokines, particularly IL-6, IL-1-beta and TNF-alpha, and whose maintenance and survival are dependent on interleukin-23 (IL-23) (Langrish, C. L. et al. (2005), J. Exp. Med. 201:233-240; Harrington, L. E., et al., (2005), Nat. Immunol. 6:1123-1132; Veldhoen, M. et al., (2006) Immunity 24:179-189). IL-23 is a heterodimeric cytokine comprised of two subunits: p19, which is unique to IL-23; and p40, which is shared with IL-12. IL-23 mediates signaling by binding to a heterodimeric receptor, comprised of IL-23R and IL-12Rbeta1 (IL12RB1), which is shared by the IL-12 receptor. Studies in murine disease models suggest that IL-23-dependent Th17 cells play a pathogenic role in autoimmune and chronic inflammatory diseases (Langrish et al., supra, Park, H., et al. (2005), Nat. Immunol. 6:1133-1141).

IL-17A is present in RA synovial fluid at the earliest stages of the disease along with other known inflammatory mediators such as TNF and IL-1β. Dys-regulated IL-17A expression within an inflamed joint singly, and in synergy with TNF and IL-1β, stimulates multiple downstream proteases, chemokines and pro-inflammatory cytokines that collectively contribute to cartilage and bone erosion. A variety of IL-17A biological antagonists used in multiple rodent arthritis models have demonstrated that IL-17A blockade inhibits arthritis progression and the resulting joint destruction that occurs with a special emphasis on bone sparing (see, e.g., Koenders M I, et al., (2006) Ann. Rheum. Dis. 65 (Suppl. 3):29-33). At least one anti-IL-17A antibody is being tested in clinical trials of human RA patients.

Currently, assessing the effect of anti-rheumatic drugs on the progression of joint destruction relies mainly on radiographic evaluation. However, how to use radiographic data in clinical trials is controversial (van der Heijde, D. et al, (2002) Arthritis Rheum 47; 215-218). In addition to being time consuming, radiography is impractical in early stages of RA in which symptoms reflecting the inflammatory process often predominate over symptoms related to joint destruction (Morozzi, G., et al, (2007) Clin Rheumatol.) Indeed, nonsteroidal anti-inflammatory drugs (NSAIDs), which have traditionally been used as first line therapies for RA, are reasonably effective at ameliorating the signs and symptoms of inflammation, but have little efficacy in retarding joint destruction, leading to speculation that inflammation and subsequent joint destruction can be uncoupled (van den Berg, WB (2001), Semin Arthritis Rheum. 30:7-16; Geusens, P. P., et al. (2006), Arthritis & Rheumatism 54 (6):1772-1777). Thus, there is a well-established clinical need for better tools to predict the effect of anti-rheumatic drugs on structural joint damage, with recent development efforts focused on various markers of cartilage and/or bone metabolism that are elevated in the serum or urine of RA patients compared to normal subjects (Crnkic, M. et al., (2003) Arthritis Res. Ther. 5:R181-R185; Valleala, H., et al. (2003) Eur. J. Endocrinol. 148:527-530); Ziolkowska, M., et al. (2002) Arthritis & Rheumatism 46(7):1744-1753).

Articular cartilage in the joints is composed of the proteoglycan aggrecan, collagen (three α-chains form a triple helix), and other non-collagenous proteins (e.g., cartilage oligomer matrix protein (COMP) and human cartilage glycoprotein-39 (HC gp-39), which is also known as YKL-40). Type I collagen is a major component of bone and other tissues; whereas, type II collagen is specifically localized to articular cartilage of the joint. At the ends of type I and II collagen helixes are short, non-helical N- and C-terminal telopeptides containing covalent cross-links that connect to other α-chains, both within the same trimer and to adjacent trimers. Physiological and pathological cleavage of collagen by MMPs or Cathepsin K results in the generation of degradation products or neo-epitopes (e.g. C2C, C1,2C, C-terminal cross-linking telopeptide of type I collagen (CTX-I), C-terminal cross-linking telopeptide of type II collagen (CTX-II), N-terminal cross-linking telopeptide of type I collagen (NTX-I)), which are released into the synovial fluid, serum, and urine. Cleavage of the collagen triple helix also releases non-collagenous proteins (e.g. COMP, YKL-40, aggrecan) previously incorporated in the collagen fibrils. These molecules are elevated in synovial fluid and serum under conditions of normal remodeling and pathological cartilage destruction.

Cartilage destruction also results in compensatory increased collagen synthesis by chondrocytes. Type I and II collagen is synthesized as a pro-molecule and once outside the cell, cleavage of pro-collagen releases N-terminal and C-terminal pro-peptides. Type II collagen C-terminal pro-peptide (CPII) levels correlate with new type II collagen synthesis. Cartilage destruction also increases aggrecan synthesis and the appearance of the “fetal form” of aggrecan that has the CS846 epitope. Increased CS846 levels in the serum reflect new aggrecan synthesis (versus cleavage of “old” aggrecan).

Bone destruction occurs via the generation of excessive numbers of osteoclasts that resorb the mineralized bone and degrade the organic matrix of the de-mineralized bone. Receptor activator of NFκB (RANK) ligand (RANKL) is a cell-surface molecule expressed by activated T-cells, fibroblast-like synoviocytes (FLS), and osteoblasts that is critical in promoting the differentiation of pre-osteoclasts into mature osteoclasts, which are cells that can erode bone. RANKL can be shed by proteolytic cleavage (both cell surface and soluble RANKL are active), and is elevated in mouse arthritis and human RA serum. Membrane or soluble RANKL binds to RANK on pre-osteoclasts and delivers a differentiation signal. Osteoprotegrin (OPG) is a natural antagonist of this system by binding to RANKL and preventing its interaction with RANK on pre-osteoclasts.

Tartrate-resistant acid phosphatase (TRACP) isoform 5b is released into the serum by bone-resorbing osteoclasts as they transcytose degraded bone proteins from the resorbed bone surface to outside the bone. TRACP isoform 5b serum levels are elevated in bone resorption diseases. The amino acid sequence for TRACP5 is found in Accession No. for NM_{—}001102405, NM_{—}001102404 or NM_{—}007388.

Some of these serum markers of bone and cartilage metabolism (or destruction) are elevated in RA patients and have some prognostic value in identifying patients that are at a higher risk of having more aggressive bone destruction. For example, elevated levels of cartilage oligomeric matrix protein (COMP) have been associated with more aggressive radiographic progression (den Broeder, A. A., et al. (2002) Ann Rheum Dis 61(4): 311-318; Wollheim, F. A., et al. (1997) Br J Rheumatol 36(8): 847-8499; Skoumal, M., et al. (2003). Scand J Rheumatol 32(3):156-161; Mansson, B., et al. (1995), J Clin Invest 95(3):1071-1077; Lindqvist, E., et al. (2005) Ann Rheum Dis 64(2):196-201; Forslind, K., et al. (1992) Br J Rheumatol 31(9): 593-598; Fex, E., et al. (1997) Br J Rheumatol 36(11):1161-1165. Also, a low OPG/RANKL ratio predicted increased five year radiographic progression (Geusens, P. P. et al. (2006) Arthritis Rheum 54(6):1772-1777) and elevated CTX-I and CTX-II levels were associated with four year Sharp Score increase in early RA patients (Garnero, P., et al. (2002) Arthritis Rheum 46(11):2847-2856).

However, the inventors herein are not aware of any published studies that conclude that blocking IL-17A can inhibit bone erosion and modulate serum levels of any of the above proteins in severely arthritic animals. Thus, a need exists to identify biomarkers that correlate with inhibition of joint destruction by anti-IL-17A therapy.

### SUMMARY OF THE INVENTION

The present invention is based on the discovery described herein that COMP, OPG and RANKL serum levels in mice with collagen-induced arthritis (CIA) following treatment with an anti-IL-17A monoclonal antibody (Mab) are modulated by anti-IL-17A therapy. Also, the inventors have discovered that RANKL serum levels in CIA-mice decrease with increasing doses of the anti-IL-17A Mab, and reach normal levels at antibody doses that are effective at inhibiting joint destruction in the CIA mice as measured by traditional histological and μ-CT-based techniques. Based on these results with COMP, OPG and RANKL in the mouse CIA arthritis model, the inventors herein believe that these markers are likely to be useful as surrogate markers, i.e., biomarkers, of the effect of anti-IL-17A therapy on joint destruction in inflammatory joint diseases such as RA and associated athritidies. Also, these data obtained in arthritic mice support the use of other markers of cartilage and bone metabolism that are elevated in human RA patients, including CTX-I, CTX-II, and HC gp-39, as surrogate markers for monitoring the effect of anti-IL-17A therapy on joint destruction in patients with inflammatory joint disease.

In addition, it has been previously discovered that anti-IL-17A therapy is effective at inhibiting joint destruction in the CIA arthritis model, even when that therapy produces no apparent improvement in inflammation (WO 2008/021156). Thus, it is expected that anti-IL-17A therapy will be useful to inhibit ongoing bone erosion in human patients who have been previously treated with a different anti-rheumatic agent, regardless of whether or not the previous agent had reduced the signs and symptoms of inflammation. Thus, the present discovery of non-inflammation related markers that are correlated with inhibition of bone erosion by IL-17A therapy provides novel methods and products for treating patients for bone erosion who are inflammatory nonresponders or inflammatory responders to previous anti-rheumatic therapy.

Accordingly, one aspect of the present invention is a method of selecting a patient with an inflammatory joint disease for treatment with an IL-17A antagonist. The patient selection method comprises comparing the level(s) of at least one joint destruction biomarker in a serum sample taken from the subject with the normal range of serum levels for the biomarker and selecting the patient for treatment with the IL-17A antagonist if the level(s) of the joint destruction biomarker in the subject's serum sample is outside of the normal range.

In another aspect, the invention provides a method of predicting efficacy of an IL-17A antagonist in inhibiting bone erosion in a subject with an inflammatory joint disease. This efficacy prediction method comprises determining the levels of at least one joint destruction biomarker in serum samples taken from the subject prior to and at the end of an initial treatment period with the IL-17A antagonist, and comparing the levels of the biomarker in these pre-treatment and post-treatment serum samples. A normalization in the level of the biomarker during the initial treatment period predicts that the IL-17A antagonist will likely be effective in inhibiting joint destruction in the subject. In a preferred embodiment, the prediction method further comprises determining the level of the biomarker in a third serum sample taken from the subject at the end of a subsequent treatment period with the IL-17A antagonist; if the level of the biomarker in the third serum sample is more normalized than the level of the biomarker in the second serum sample, then the IL-17A antagonist is predicted to be effective in inhibiting joint destruction in the subject. Preferred initial treatment periods are at least one week, at least two weeks, at least four weeks, at least eight weeks, at least twelve weeks, at least 24 weeks, or at least 48 weeks, while preferred subsequent treatment periods are at least 12 weeks, at least 24 weeks or at least 48 weeks. In some embodiments, the subject is a non-human animal with arthritis, which may be naturally present or experimentally-induced. Preferred non-human subjects include CIA mice or rats with adjuvant-induced arthritis (AIA). In other embodiments, the subject is a human with arthritis.

In yet a further aspect, the present invention provides a method of treating a subject for an inflammatory joint disease with an IL-17A antagonist. This treatment method comprises determining, in a first serum sample taken from the subject, the level of at least one joint destruction biomarker, administering the IL-17A antagonist to the subject according to a first dosing regimen during an initial treatment period, determining the level of the selected biomarker(s) in at least a second serum sample taken from the patient at the end of the initial treatment period, and comparing the levels of the biomarker in the first and second serum samples.

If the level of the biomarker in the second serum sample is within a specified range, then the subject is treated with the IL-17A antagonist according to the first dosing regimen during at least one subsequent treatment period. However, if the level of the biomarker in the second serum sample is outside of the specified range, e.g., indicating that more aggressive therapy may be necessary to achieve inhibition of joint destruction, then the subject is treated with the IL-17A antagonist according to a second dosing regimen during at least one subsequent treatment period, wherein the second dosing regimen comprises administering a total amount of the IL-17A antagonist during the subsequent treatment period that is greater than the total amount administered during the initial treatment period.

In one preferred embodiment of this treatment method, the specified range is the normal range, i.e., the range of serum levels of the biomarker found in a population of healthy, gender- and age-matched subjects. In another preferred embodiment, the specified range for the serum level of the biomarker is defined by a confidence interval of at least 80% of the mean serum level of the biomarker in a population of subjects with the inflammatory disease who were treated with the same IL-17A antagonist according to the same dosing regimen for a time period equal to or longer than the initial treatment period, wherein the population exhibited inhibition of joint destruction following treatment with the IL-17A antagonist according to the first dosing regimen during a time period equal to or longer than the subsequent treatment period.

In one preferred embodiment, when the biomarker level in the second serum sample indicates that more aggressive anti-IL-17A therapy is required, the subject is treated with a greater total amount of the antagonist during the subsequent treatment period(s) by administering the IL-17A antagonist at higher doses and/or at more frequent intervals than the doses and intervals employed during the initial treatment period.

In another preferred embodiment, the treatment method further comprises administering an IL-23 antagonist during each of the initial treatment and subsequent treatment periods, or during only a subsequent treatment period. The IL-23 antagonist may inhibit the expression of either subunit of the cytokine (IL-23p19 or p40), either subunit of the functional receptor (IL-23R or IL-12beta1), or may inhibit IL-23 signaling by directly or indirectly interacting with one or more of these polypeptides to prevent a functional ligand-receptor interaction. In some preferred embodiments, the IL-23 antagonist is an antibody or antibody fragment that binds to and inhibits the activity of either IL-23p19 or IL-23R. In one particularly preferred embodiment, the IL-23 antagonist is a monoclonal antibody that specifically binds to IL-23p19.

The invention also provides a kit for treating an inflammatory joint disease. The kit comprises a pharmaceutical composition of an IL-17A antagonist and reagents for measuring the level of at least one joint destruction biomarker in a serum sample taken from a subject.

The invention also provides a manufactured drug product for treating an inflammatory joint disease, which comprises a pharmaceutical formulation comprising an IL-17A antagonist and instructions for determining patient serum levels of at least one joint destruction biomarker before and during treatment with the IL-17A antagonist.

Yet another aspect of the invention is the use of an IL-17A antagonist for preparing a medicament for treating a patient having an inflammatory joint disease to inhibit joint destruction, wherein the patient has an abnormal serum level of at least one joint destruction biomarker after previous treatment with a different anti-rheumatic therapy. In a preferred embodiment, the medicament is for administering the IL-17A antagonist according to any of the treatment regimens described herein.

In each of the above described aspects of the invention, the IL-17A antagonist may inhibit the expression of IL-17A or IL-17RA or IL-17RC or may inhibit IL-17A signaling by directly or indirectly interacting with one or more of these polypeptides to prevent a functional ligand-receptor interaction. In some preferred embodiments, the IL-17A antagonist is an antibody or antibody fragment that binds to and inhibits the activity of either IL-17A, IL-17RA or IL-17RC. In one particularly preferred embodiment, the IL-17A antagonist is a monoclonal antibody that specifically binds to IL-17A. In other preferred embodiments, the IL-17A antagonist is a bispecific antibody that binds to and inhibits the activity of IL-23p19 and IL-17A; IL-23p19 and IL-17RA; IL-23R and IL-17A; IL-23R and IL-17RA, IL-23p19 and IL-17RC; or IL-23R and IL-17RC. In another particularly preferred embodiment, the IL-17A antagonist is a bispecific antibody that binds to and inhibits the activity of IL-23p19 and IL-17A.

In each of the above-described aspects of the invention, preferred joint destruction biomarkers are COMP, CTX-I, CTX-II, HC-39, OPG, RANKL, TRACP) isoform 5b and osteocalcin. Any one of these biomarkers or any combination of two or more, or all six, of these biomarkers may be employed. COMP, OPG and RANKL are more preferred biomarkers, with RANKL being the most preferred biomarker.

Preferred inflammatory joint diseases that may be treated using any of the above aspects of the invention are rheumatoid arthritis (RA), psoriatic arthritis (PsA) or ankylosing spondylitis (AS), with rheumatoid arthritis being a particularly preferred inflammatory joint disease.

Also, in each of the above-described aspects of the invention, the subject with the inflammatory joint disease may be one that is an inflammatory nonresponder, an inflammatory responder, a moderate inflammatory responder, or a good inflammatory responder to the IL-17A antagonist or a different anti-rheumatic drug.

### BRIEF DESCRIPTION OF THE DRAWINGS

FIG. 1A shows the amino acid sequence of the light chain of humanized anti-IL-17A antibody 16C10 according to the present invention SEQ ID NO:1. CDRs are indicated.

FIG. 1B shows amino acid sequence for the heavy chain of humanized anti-IL-17A antibody 16C10 according to the present invention SEQ ID NO: 2. CDRs are indicated.

FIGS. 2A-2D shows the effects of anti-IL-17A antibody treatments on pathology in the CIA mouse model of rheumatoid arthritis. Treatments include administration of anti-IL-17A antibody JL7.1D10 (at 28, 7, and 2 mg/kg) and administration of an isotype control (7 mg/kg).

FIG. 2A presents visual disease severity score (DSS), a measure of visual paw swelling and redness, as a function of antibody treatment. Scoring is: 0=paw appears the same as control (untreated) paw; 1=inflammation of one finger on a given paw; 2=inflammation of two fingers or the palm of a given paw; 3=inflammation of the palm and finger(s) of a given paw.

FIG. 2B presents cartilage damage (by histopathology) as a function of antibody treatment. Scoring is: 0=normal; 1=minimal, 2=mild; 3=moderate; 4=severe.

FIG. 2C presents bone erosion (by histopathology) as a function of antibody treatment. Scoring is: 0=normal; 1=minimal, 2=mild; 3=moderate; 4=severe.

FIG. 2D presents bone erosion (by histopathology) for paws from CIA mice that scored 2 or 3 in visual DSS, i.e. highly inflamed paws. rIgG1 is an isotype control antibody. Scoring is: 0=normal; 1=minimal, 2=mild; 3=moderate; 4=severe.

FIGS. 3A-3B presents data showing the effect of an anti-IL-17A antibody on serum COMP levels in arthritic mice.

FIG. 3A presents serum COMP levels in mice treated with isotype control or an anti-IL-17A antibody (JL7.1D10). The solid horizontal bar denotes the average serum COMP level in non-diseased animals (grey circles on left side of graphs) and the two dotted horizontal bars denotes +/−2 standard deviations from the non-diseased mice average.

FIG. 3AA presents serum COMP levels in non-diseased (normal) mice or in CIA mice treated weekly for five weeks with an isotype control or an anti-IL-17A antibody (JL7.1D10) at a dose of 28 mg/kg or 7 mg/kg.

FIG. 3B presents serum COMP levels in non-diseased (un-manipulated) and severely arthritic mice treated with short term isotype control (rIgG1) or an anti-IL-17A antibody (JL7.1D10).

FIG. 4 presents serum RANKL levels in arthritic mice who were untreated, treated with an isotype control, or treated with one of three doses of an anti-IL-17A antibody (JL7.1D10). The solid horizontal bar denotes the average serum COMP level in non-diseased animals (grey circles on left side of graphs) and the two dotted horizontal bars denotes +/−2 standard deviations from the non-diseased mice average.

FIG. 5 presents serum OPG levels in normal mice (grey circles) or in arthritic mice who were untreated (no dosing), treated with an isotype control (Rat IgG1), or treated with an anti-IL-17A antibody.

FIG. 6 presents serum RANKL and OPG levels in normal mice (grey circles) or in severely arthritic mice following short-term exposure to an isotype control (rIgG1) or an anti-IL-17A antibody (JL7.1D10).

FIG. 7 presents micro-CT X-rays of an un-inflamed mice paw, and of severely inflamed paws from arthritic mice treated with an isotype control antibody or an anti-IL-17A antibody.

FIG. 8 presents serum TRACP levels in non-diseased (un-manipulated) and severely arthritic mice treated with long term isotype control (rIgG1) or an anti-IL-17A antibody (JL7.1D10).

FIG. 9 presents serum osteocalcin levels in normal mice and in CIA mice treated with an anti-IL-17A antibody (Th7.1D10) or an isotype control antibody

FIG. 10 shows the dynamic change in serum RANKL (left panel) and OPG (right panel) levels in a cohort of mice progressing through the mouse CIA mode, with horizontal lines indicating mean (unbroken line) +/−S.D. (broken lines) in un-manipulated healthy mice and antibody dosing denoted as arrowheads.

FIG. 11 shows the serum RANKL concentration versus total animal DSS from individual normal or CIA mice at each of five weeks of treatment with a rat IgG1 isotype control antibody.

FIG. 12 shows the serum OPG concentration versus total animal DSS from individual normal or CIA mice at each of five weeks of treatment with a rat IgG1 isotype control antibody.

FIG. 13 presents the serum RANKL levels in individual CIA mice that were not treated or treated with five weekly subcutaneous doses of an isotype control antibody (rat IgG1) or an anti-IL-17A antibody (JL7.1D10) at 2, 7, or 28 mg/kg.

FIG. 14 shows the serum OPG profiles for individual CIA mice that were not treated or treated with five weekly subcutaneous doses of 7 mg/kg of an isotype control antibody (rat IgG1) or 28 mg/kg of an anti-IL-17A antibody (JL7.1D10).

FIG. 15 shows the thickness of paws in rats with adjuvant-induced arthritis (AIA) that were treated prior to adjuvant injection with an isotype antibody or with the indicated doses of an anti-rat IL-17A antibody (JL8.18E10).

FIG. 16 illustrates the effect of anti-IL-17A or anti-TNF therapy on serum RANKL levels at days 14 and 25 following drug exposure in individual rats with established AIA.

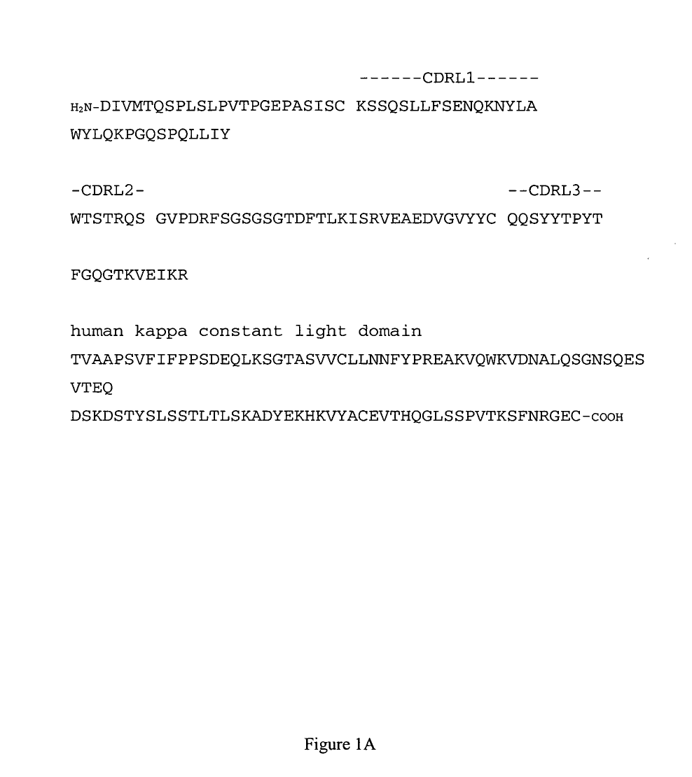

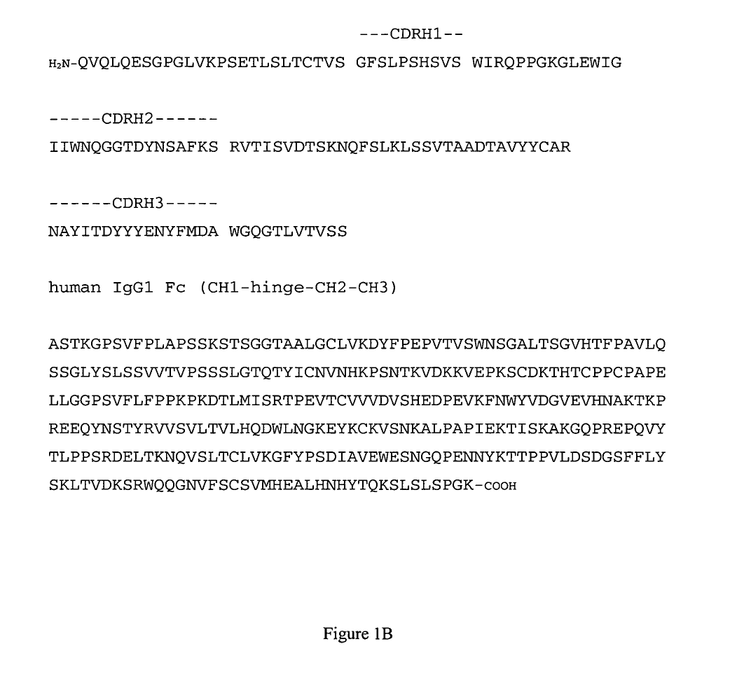

### DETAILED DESCRIPTION

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Abnormal” in the context of the serum level of a joint destruction biomarker means that the serum level is outside of the normal range for that biomarker.

“Ankylosing spondylitis” or “AS” is a form of chronic inflammation of the spine and the sacroiliac joints, which are located in the low back where the sacrum (the bone directly above the tailbone) meets the iliac bones (bones on either side of the upper buttocks). Chronic inflammation in these areas causes pain and stiffness in and around the spine. Over time, chronic spinal inflammation (spondylitis) can lead to a complete cementing together (fusion) of the vertebrae, a process referred to as ankylosis. Ankylosis leads to loss of mobility of the spine. Ankylosing spondylitis is also a systemic rheumatic disease, meaning it can affect other tissues throughout the body. Accordingly, it can cause inflammation in or injury to other joints away from the spine, as well as other organs, such as the eyes, heart, lungs, and kidneys.

“Antagonist” means any molecule that can prevent, neutralize, inhibit or reduce a targeted activity, i.e., the activity of a cytokine such as IL-17A, either in vitro or in vivo. Cytokine antagonists include, but are not limited to, antagonistic antibodies, peptides, peptide-mimetics, polypeptides, and small molecules that bind to a cytokine (or any of its subunits) or its functional receptor (or any of its subunits) in a manner that interferes with cytokine signal transduction and downstream activity. Examples of peptide and polypeptide antagonists include truncated versions or fragments of the cytokine receptor (e.g., soluble extracellular domains) that bind to the cytokine in a manner that either reduces the amount of cytokine available to bind to its functional receptor or otherwise prevents the cytokine from binding to its functional receptor. Antagonists also include molecules that prevent expression of any subunit that comprises the cytokine or its receptor, such as, for example, antisense oligonucleotides which target mRNA, and interfering messenger RNA, (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189). The inhibitory effect of an antagonist can be measured by routine techniques. For example, to assess the inhibitory effect on cytokine-induced activity, human cells expressing a functional receptor for a cytokine are treated with the cytokine and the expression of genes known to be activated or inhibited by that cytokine is measured in the presence or absence of a potential antagonist. Antagonists useful in the present invention inhibit the targeted activity by at least 25%, preferably by at least 50%, more preferably by at least 75%, and most preferably by at least 90%, when compared to a suitable control.

“Antibody” refers to any form of antibody that exhibits the desired biological activity, such as inhibiting binding of a ligand to its receptor, or by inhibiting ligand-induced signaling of a receptor. Thus, “antibody” is used in the broadest sense and specifically covers, but is not limited to, monoclonal antibodies (including full length monoclonal antibodies), polyclonal antibodies, and multispecific antibodies (e.g., bispecific antibodies).

“Antibody fragment” and “antibody binding fragment” mean antigen-binding fragments and analogues of an antibody, typically including at least a portion of the antigen binding or variable regions (e.g. one or more CDRs) of the parental antibody. An antibody fragment retains at least some of the binding specificity of the parental antibody. Typically, an antibody fragment retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. Examples of antibody fragments include, but are not limited to, Fab, Fab′, F(ab′)₂, and Fv fragments; diabodies; linear antibodies; single-chain antibody molecules, e.g., sc-Fv; and multispecific antibodies formed from antibody fragments. Engineered antibody variants are reviewed in Holliger and Hudson (2005) Nat. Biotechnol. 23:1126-1136.

A “Fab fragment” is comprised of one light chain and the C_{H}1 and variable regions of one heavy chain. The heavy chain of a Fab molecule cannot form a disulfide bond with another heavy chain molecule.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” comprises the variable regions from both the heavy and light chains, but lacks the constant regions.

A “single-chain Fv antibody (or “scFv antibody”) refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448.

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

“Anti-rheumatic drug” is a drug used to treat rheumatoid arthritis. The major classes of anti-rheumatic drugs are described below.

“Nonsteroidal Anti-Inflammatory Drugs” or “NSAIDs” are drugs with analgesic, antipyretic and anti-inflammatory effects—they reduce pain, fever and inflammation. NSAIDs are used to provide symptomatic relief in RA, but have a limited effect on the progressive bone and cartilage loss associated with rheumatoid arthritis. NSAIDs include salicylates, arlyalknoic acids, 2-arylpropionic acids (profens), N-arylanthranilic acids (fenamic acids), oxicams, coxibs, and sulphonanilides. Common NSAIDs include: ibuprofen, naproxen and indomethacin.

“Corticosteroids” are synthetic analogs of cortisone and are used to reduce inflammation and suppress activity of the immune system. The most commonly prescribed are prednisone and dexamethasone.

“Disease Modifying Anti-Rheumatic Drugs” or “DMARDs” are drugs which influence the disease process itself. DMARDs, which are also known as remittive drugs, also have anti-inflammatory effects, and most were borrowed from the treatment of other diseases, such as cancer and malaria. DMARDs include chloroquine, hydroxychloroquine, methotrexate, sulfasalazine, cyclosporine, azathioprine and cyclophosphamide, azathioprine, sulfasalazine, penicillamine, and organic gold compounds such as aurothioglucose, gold sodium thiomalate and auranofin. DMARDs also include agents directed against pro-inflammatory cytokines and their receptors, such TNF-alfa inhibitors. Examples of TNF-antagonists that have been approved for treating RA, include Infliximab (Remicade®, Centocor, Malvern, Pa.), Etanercept (Enbrel®, Amgen, Thousand Oaks, Calif.), and Adalimumab (Humira®, Abbott Laboratories, Abbott Park, Ill.). IL-17A antagonists also would be classified as DMARDs.

“Slow-Acting Anti-rheumatic Drugs” or “SAARDs” are a special class of DMARDs and the effect of these drugs is slow acting and not so quickly apparent as that of the NSAIDs. Examples of SAARDs are hydroxychloroquine and aurothioglucose.

“Immunosuppressive cytotoxic drugs” or “immunosuppressive drugs” are anti-rheumatic drugs typically used for inflammatory joint diseases if prior treatment with NSAIDs and SAARDs had no effect. Examples of immunosuppressive drugs are: methotrexate, mechlorethamine, cyclophosphamide, chlorambucil, and azathioprine.

“Binding compound” refers to a molecule, small molecule, macromolecule, antibody, a fragment or analogue thereof, or soluble receptor, capable of binding to a specified target. “Binding compound” also may refer to any of the following that are capable of binding to the specified target: a complex of molecules (e.g., a non-covalent molecular complex); an ionized molecule; and a covalently or non-covalently modified molecule (e.g., modified by phosphorylation, acylation, cross-linking, cyclization, or limited cleavage). In cases where the binding compound can be dissolved or suspended in solution, “binding” may be defined as an association of the binding compound with a target where the association results in reduction in the normal Brownian motion of the binding compound.

“Binding composition” refers to a binding compound in combination with at least one other substance, such as a stabilizer, excipient, salt, buffer, solvent, or additive.

“Bispecific antibody” means an antibody that has two antigen binding sites having specificities for two different epitopes, which may be on the same antigen, or on two different antigens. Bispecific antibodies include bispecific antibody fragments. See, e.g., Hollinger, et al. (1993) Proc. Natl. Acad. Sci. U.S.A. 90: 6444-48, Gruber, et al., J. Immunol. 152: 5368 (1994).

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition. As a nonlimiting example, a cytokine or an antibody chain which consists essentially of a recited amino acid sequence may also include one or more amino acids that do not materially affect the properties of the cytokine or the antibody chain.

“Inflammatory joint disease” means any disease or condition in which (a) inflammation is present in any joint and (b) the inflammation is part of an immune response that requires or is promoted by IL-17. Nonlimiting examples of inflammatory joint diseases include ankylosing spondylitis, psoriatic arthritis, rheumatoid arthritis:

“Inflammatory response” to an anti-rheumatic drug means a reduction in the signs and symptoms of inflammation, as measured using any accepted standard known in the art for the inflammatory joint disease of interest. For example, comparing the number of tender and swollen joints between baseline and various time points during treatment is a typical way to assess joint status and response. In the American College of Rheumatology (ACR) joint count for RA (Felson et al. (1995) Arthritis & Rheumatology 38; 727-735), 68 joints are assessed for tenderness and 66 for swelling (the hip is not assessed for swelling). In the Disease Activity Score (DAS) employed primarily in Europe, either a 44- or 28-joint count is used in RA. In PsA, most recent trials of anti-rheumatic drugs have used a 78 tender and 76 swollen joint count in order to accommodate the frequently involved distal interphalangeal and carpal metacarpal joints. In addition to the joint count, the ACR evaluation criteria include the following elements to comprise a composite score: patient global (on a visual analog scale [VAS]), patient pain, physician global, Health Assessment Questionnaire (HAQ; a measure of function), and an acute-phase reactant (either C-reactive protein or sedimentation rate). An ACR 20 response would constitute a 20% improvement in tender and swollen joint count and a 20% improvement of at least 3 of the other 5 elements in the composite criteria. ACR 50 and 70 responses represent at least a 50% and 70% improvement of these elements. The ACR system only represents change, whereas the DAS system represents both current state of disease activity and change. The DAS scoring system uses a weighted mathematical formula, derived from clinical trials in RA. For example, the DAS 28 is 0.56(√T28)+0.28(√SW28)+0.70(Ln ESR)+0.014 GH wherein T represents tender joint number, SW is swollen joint number, ESR is erythrocyte sedimentation rate, and GH is global health. Various values of the DAS represent high or low disease activity as well as remission, and the change and endpoint score result in a categorization of the patient by degree of response (none, moderate, good).

“Inflammatory responder” to an anti-rheumatic drug means a subject who, after 3 months of treatment with the drug, has at least a >20% improvement over baseline (e.g., pre-treatment) in ACR tender joint count and at least a >20% improvement over baseline in ACR swollen joint count.

“Good inflammatory responder” means a subject who has at least a 70% improvement over baseline in each of ACR tender and swollen joint counts after 3 months treatment with the drug.

“Moderate inflammatory responder” means a subject who has at least a 50% improvement over baseline in each of ACR tender and swollen joint counts after 3 months treatment with the drug.

“Inflammatory nonresponder” to an anti-rheumatic drug means a subject who, after 3 months of treatment with the drug, has either ≦20% improvement over baseline in ACR tender joint count or ≦20% improvement over baseline in ACR swollen joint count, or who fails to complete treatment with the anti-rheumatic drug for 3 months due to intolerable adverse effects or worsening of symptoms.

“Interleukin-12R beta1” or “IL12RB1” means a single polypeptide chain consisting essentially of the sequence of human IL12RB1 as described in NCBI Protein Sequence Database Accession Numbers NP714912, NP005526 or naturally occurring variants thereof.

“Interleukin-17” or “IL-17” or “IL-17A” means a protein consisting of one or two polypeptide chains, with each chain consisting essentially of (1) the sequence of human IL17A as described in any of NCBI Protein Sequence Database Accession Numbers NP002181, AAH67505, AAH67503, AAH67504, AAH66251, AAH66252, or (2) naturally occurring variants of these sequences, including the mature form of the polypeptide chain, i.e., lacking the signal peptide, or (3) the sequence of a non-human IL-17A, including mice IL-17A or rat IL-17A.

“IL-17R” or “IL-17RA” means a single polypeptide chain consisting essentially of the sequence of human IL-17RA as described in WO 96/29408 or in any of NCBI Protein Sequence Database Accession Numbers: NP 055154, Q96F46, CAJ86450, or naturally occurring variants of these sequences, including the mature form of the polypeptide chain, i.e., lacking the signal peptide.

“IL-17RC” means a single polypeptide chain consisting essentially of the sequence of human IL-17RC as described in WO 238764A2 or in any of NCBI Protein Sequence Database Accession Numbers NP703191, NP703190 and NP116121, or naturally occurring variants of these sequences, including the mature form of the polypeptide chain, i.e., lacking the signal peptide.

“Interleukin-23 (or “IL-23) means a protein consisting of two polypeptide chains. One chain consists essentially of the sequence of human IL23, subunit p19 (also known as IL23A) as described in any of NCBI Protein Sequence Database Accession Numbers NP057668, AAH67511, AAH66267, AAH66268, AAH66269, AAH667512, AAH67513 or naturally occurring variants of these sequences, including the mature form of the polypeptide chain, i.e., lacking the signal peptide. The other chain consists essentially of the sequence of human IL12, subunit p40 (also known as IL12B and IL23, subunit p40) as described in any of NCBI Protein Sequence Database Accession Numbers NP002178, P29460, AAG32620, AAH74723, AAH67502, AAH67499, AAH67498, AAH67501 or naturally occurring variants of these sequences, including the mature form of the polypeptide chain, i.e., lacking the signal peptide.

“Interleukin-23R” or “IL-23R” means a single polypeptide chain consisting essentially of the sequence of human IL23R as described in NCBI Protein Sequence Database Accession Number NP653302 or naturally occurring variants thereof, including the mature form of the polypeptide chain, i.e., lacking the signal peptide.

“Joint” means the area where two bones are attached for the purpose of motion of body parts. A joint is usually formed of fibrous connective tissue and cartilage. An articulation or an arthrosis is the same as a joint.

“Monoclonal antibody” or “mAb” means an antibody obtained from a substantially homogeneous population of antibodies, and is not to be construed as requiring production of the antibody by any particular method.

“Normal range” in the context of serum biomarker levels refers to the range of serum levels of the biomarker found in a population of healthy, gender- and age-matched subjects. The minimal size of this healthy population may be determined using standard statistical measures, e.g., the practitioner could take into account the incidence of the disease in the general population and the level of statistical certainty desired in the results. Preferably, the normal range for serum levels of a joint destruction biomarker is determined from a population of at least five, ten or twenty subjects, more preferably from a population of at least forty or eighty subjects, and even more preferably from more than 100 subjects.

“Normalizes” or “Normalization” in the context of serum biomarker levels refers to an up or down change in the serum level of a biomarker following treatment with an IL-17A antagonist such that the changed serum level is closer to the normal range for that biomarker or preferably falls within the normal range. The levels of some serum markers of bone and cartilage metabolism or destruction are increased in arthritic subjects (e.g., COMP and RANKL); thus for such markers a normalization means that the serum level is decreased following treatment with an IL-17A antagonist, compared to the serum level prior to treatment. However, other serum markers of bone and cartilage metabolism or destruction are decreased in arthritic subjects (e.g., osteocalcin); thus, for such markers a normalization means that the serum level is increased following treatment with an IL-17A antagonist, compared to the serum level prior to such treatment.

“Parenteral administration” means an intravenous, subcutaneous, or intramuscular injection.

“Small molecule” means a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. Peptide mimetics of antibodies and cytokines are known in the art. See, e.g., Casset, et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999) Nat. Struct. Biol. 6:652-656; Sato and Sone (2003) Biochem. J. 371:603-608; U.S. Pat. No. 6,326,482 issued to Stewart, et al.

“Psoriatic arthritis” or “PsA” is a chronic disease characterized by inflammation of the skin (psoriasis) and joints (arthritis). Psoriasis features patchy, raised, red areas of skin inflammation with scaling and often affects the tips of the elbows and knees, the scalp, the navel, and around the genital areas or anus. Approximately 10% of patients who have psoriasis also develop an associated inflammation of their joints. Patients who have both inflammatory arthritis and psoriasis are diagnosed as having psoriatic arthritis. Psoriatic arthritis is a systemic rheumatic disease that can also cause inflammation in body tissues away from the joints and the skin, such as in the eyes, heart, lungs, and kidneys.

“Serum” means blood serum or blood plasma.

“Subject” means any animal. In some preferred embodiments, it will be readily apparent to the skilled artisan from the context that the subject is a research animal such as a rodent, including mice or arts with or without experimentally induced arthritis. In other preferred embodiments, it will be readily apparent to the skilled artisan that the subject is a human.

“Treat” or “Treating” means to administer a therapeutic agent, such as a composition containing any of the IL-17A antagonists described herein, internally or externally to a patient in need of the therapeutic agent. Typically, the agent is administered in an amount effective to prevent or alleviate one or more disease symptoms, or one or more adverse effects of treatment with a different therapeutic agent, whether by preventing the development of, inducing the regression of, or inhibiting the progression of such symptom(s) or adverse effect(s) by any clinically measurable degree. The amount of a therapeutic agent that is effective to alleviate any particular disease symptom or adverse effect (also referred to as the “therapeutically effective amount”) may vary according to factors such as the disease state, age, and weight of the patient, and the ability of the therapeutic agent to elicit a desired response in the patient. Whether a disease symptom or adverse effect has been alleviated can be assessed by any clinical measurement typically used by physicians or other skilled healthcare providers to assess the severity or progression status of that symptom or adverse effect. When a therapeutic agent is administered to a patient who has active disease, a therapeutically effective amount will typically result in a reduction of the measured symptom by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. While an embodiment of the present invention (e.g., a treatment method or drug product) may not be effective in preventing or alleviating the target disease symptom(s) or adverse effect(s) in every patient, it should alleviate such symptom(s) or effect(s) in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. General

As described in more detail in the Examples below, the present invention is based on the discoveries that anti-IL-17A therapy in a mouse model of RA (1) inhibits bone erosion, even when the anti-IL-17A therapy did not have an apparent effect on inflammation and (2) decreases serum levels of several markers of cartilage and/or bone metabolism. These discoveries have the following implications for anti-IL-17A therapy of human inflammatory joint diseases:
patients whose outward signs of disease (e.g. tender and swollen joint counts, serum IL-6, seurm CRP, acute phase reactants, HAQ, patient and physician global assessments, ACR20/50/70 composite scoring system) are not adequately being controlled by other anti-rheumatic drugs may derive joint protective benefit from blocking IL-17A;
patients treated with an IL-17A antagonist who derive little to no benefit as assessed by these outward signs of disease may still be achieving inhibition of joint destruction as assessed by X-ray (measure of bone erosion) or MRI (measure of synovial inflammation);
patients having serum levels of disease markers that pronostically suggest the potential for accelerated joint destruction (e.g., elevated serum levels of COMP, CTX-II, CTX-I, RANKL, OPG, TRACP, YKL-40, or other cartilage and/or bone destruction markers, or reduced serum levels of osteocalcin) may experience the most joint-preserving benefit from anti-IL-17A therapy, regardless of whether such therapy modulates the outward signs of disease; and
short term modulation of the elevated levels of serum markers of bone and cartilage destruction (e.g., COMP, CTX-I, CTX-II, HC gp-39, OPG and RANKL) or depressed serum makers of bone destruction (e.g., osteocalcin) can be used to assess whether IL-17A blockade holds long term promise as a joint protective therapy, i.e. as pharmacodynamic markers or surrogate markers for X-ray-based efficacy measures. Thus, the present invention provides methods, kits and drug products that are directed to the use of joint destruction biomarkers to guide therapy with IL-17A antagonists.

Measurement of the serum level of a joint destruction biomarker employed in the present invention may be achieved using any technique known in the art. Assays for COMP, CTX-I, CTX-II, HC gp-39, OPG and RANKL are either commercially available or described in the literature.

COMP assays: AnaMar Medical, Lund, Sweden (Crnkic, M., et al., Arth. Res. Ther. 2003; 5:R181-R185); AnaMar Medical, Lund, Sweden (Mundermann, A., et al., Osteo and Cartilage 2005; 13:34-38); AnaMar Medical, Lund, Sweden (Skoumal, M., et al., Arth. Res. Ther. 2004; 6:73-74); AnaMar Medical, Lund, Sweden (Skoumal Scand J. Rheum. 2003); AnaMar Medical, Lund, Sweden (Lindqvist, E., et al., Ann. Rheum. Dis 2005; 64:196-201); Lab Inhibition ELISA (Wislowska, Clin. Rheumatol 2005; 24:278-284); Lab ELISA (Mansson, J. Clin. Invest. 1995); Lab ELISA (Senolt Physiological Res. 2007); Ana Mar Medical, Lund, Sweden (Morozzi, G., et al., Clin Rheum 2007); AnaMar Medical, Lund, Sweden (Andersson, M. L. E., et al., Ann Rheum Dis 2006; 65:1490-1494).

HC gp39 Assays: Quidel, US (Hetland, ACR2006); Lab assay (den Broeder, A. A., et al., Ann Rheum Dis 2002; 61:311-318); Lab RIA assay (Johansen, J. S., et al., Rheumatology 1999; 38:618-626).

RANKL Assays: AMGEN in-house ELISA (Geusens, P. P., et al., Arth Rheum 2006; 54(6):1772-1777); ELISA (Immun-diagnostik, Germany, Vis Ard.bmjjournals.com 2006).

OPG Assays: Biomedica Medizinprodukte, Vienna, Austria (Geusens, Arth Rheum 2006; supra); ELISA (Immundiagnostik, Germany) (V is Ard.bmjjournals.com 2006); ELISA (Immunodiagnostik, Bensheim, Germany); (Valleala, H., et al., Eur. J. Endocrinology 2003; 148:527-530).

CTX-I Assays: CrossLaps ELISA (Osteometer Biotech, Herlev, Denmark) (Garnero, P., et al., Arth Rheuma 2002; 46(11):2847-2856); CrossLaps (Rosch, Mannheim, Germany) (Lange, U., et al., Rheumatology 2005; 44:1546-1548).

CTX-II Assays: CartiLaps ELISA (Osteometer Biotech, Herlev, Denmark) (Garnero, P., et al., Arth Rheuma 2002; supra); CartiLaps ELISA (Osteometer Biotech, Herlev, Denmark) (Garnero, Arth Rheuma 2002; 46:21-30); PC-Cartilaps (Nordic Biosciences Diagnostics, Herlev, Denmark) (Olsen, A. K., et al., Osteoarth. and Cartilage 2007; 15:335-342).

Antagonists useful in the present invention inhibit, block or neutralize IL-17A activity, which includes inhibiting IL-17A activity in promoting accumulation of neutrophils in a localized area and inhibiting IL-17A activity in promoting the activation of neutrophils (see, e.g., Kolls, J. et al. (2004) Immunity Vol. 21, 467-476). IL-17A can induce or promote the production of any of the following proinflammatory and neutrophil-mobilizing cytokines, depending on the cell type: IL-6, MCP-1, CXCL8 (IL-8), CXCL1, CXCL6, TNFα, IL-1β, G-CSF, GM-CSF, MMP-1, and MMP-13.

IL-17A antagonists useful in the present invention include a soluble receptor comprising the extracellular domain of a functional receptor for IL-17A. Soluble receptors can be prepared and used according to standard methods (see, e.g., Jones, et al. (2002) Biochim. Biophys. Acta 1592:251-263; Prudhomme, et al. (2001) Expert Opinion Biol. Ther. 1:359-373; Fernandez-Botran (1999) Crit. Rev. Clin. Lab Sci. 36:165-224).

Preferred IL-17A antagonists for use in the present invention are antibodies or bispecific antibodies that specifically bind to, and inhibit the activity of, any of IL-17A, IL-17RA, IL-17RC, and a heteromeric complex comprising IL-17RA and IL-17RC. More preferably, the target of the IL-17A antagonist is IL-17A or IL-17RA. Particularly preferred IL-17A antagonists specifically bind to, and inhibit the activity of IL-17A. A particularly preferred IL-17A antagonist is a humanized monoclonal antibody which comprises a light chain having SEQ ID NO:1 and a heavy chain having SEQ ID NO:2.

Another preferred IL-17A antagonist for use in the present invention is a bispecific antibody, or bispecific antibody fragment, which also antagonizes IL-23 activity. Such bispecific antagonists specifically bind to, and inhibit the activity of, each member in any of the following combinations: IL-17A and IL-23; IL-17A and IL-23p19; IL-17A and IL-12p40; IL-17A and an IL-23R/IL12RB1 complex; IL-17A and IL-23R; IL-17A and IL12RB1; IL17RA and IL-23; IL-17RA and IL-23p19; IL-17RA and IL-12p40; IL-17RA and an IL-23R/IL12RB1 complex; IL-17RA and IL-23R; IL-17RA and IL12RB1; IL17RC and IL-23; IL-17RC and IL-23p19; IL-17RC and IL-12p40; IL-17RC and an IL-23R/IL12RB1 complex; IL-17RC and IL-23R; IL-17RC and IL12RB1; an IL-17RA/IL-17RC complex and IL-23; an IL-17RA/IL-17RC complex and IL-23p19; an IL-17RA/IL-17RC complex and IL-12p40; an IL-17RA/IL-17RC complex and an IL-23R/IL12RB1 complex; an IL-17RA/IL-17RC complex and IL-23R; and an IL-17RA/IL-17RC complex and IL12RB1. Preferred combinations targeted by bispecific antibodies used in the present invention are: IL-17A and IL-23; IL-17A and IL-23p19; IL17RA and IL-23; and IL-17RA and IL-23p19. A particularly preferred bispecific antibody specifically binds to, and inhibits the activity of, each of IL-17A and IL-23p19.

Preferred IL-23 antagonists are antibodies that bind to, and inhibit the activity of, any of IL-23, IL-23p19, IL-12p40, IL23R, IL12RB1, and an IL-23R/IL12RB1 complex. Another preferred IL-23 antagonist is an IL-23 binding polypeptide which consists essentially of the extracellular domain of IL-23R, e.g., amino acids 1-353 of GenBankAAM44229, or a fragment thereof.

Antibody antagonists for use in the invention may be prepared by any method known in the art for preparing antibodies. The preparation of monoclonal, polyclonal, and humanized antibodies is described in Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; and U.S. Pat. No. 6,329,511 issued to Vasquez, et al.

Any antigenic form of the desired target can be used to generate antibodies, which can be screened for those having the desired antagonizing activity. Thus, the eliciting antigen may be a peptide containing a single epitope or multiple epitopes, or it may be the entire protein alone or in combination with one or more immunogenicity enhancing agents known in the art. To improve the immunogenicity of an antigenic peptide, the peptide may be conjugated to a carrier protein. The antigen may also be an isolated full-length protein, a cell surface protein (e.g., immunizing with cells transfected with at least a portion of the antigen), or a soluble protein (e.g., immunizing with only the extracellular domain portion of the protein). The antigen may be expressed by a genetically modified cell, in which the DNA encoding the antigen is genomic or non-genomic (e.g., on a plasmid).

A peptide consisting essentially of a region of predicted high antigenicity can be used for antibody generation. For example, regions of high antigenicity of human p19 occur at amino acids 16-28; 57-87; 110-114; 136-154; and 182-186 of GenBank AAQ89442 (gi:37183284) and regions of high antigenicity of human IL-23R occur at amino acids 22-33; 57-63; 68-74; 101-112; 117-133; 164-177; 244-264; 294-302; 315-326; 347-354; 444-473; 510-530; and 554-558 of GenBank AAM44229 (gi: 21239252), as determined by analysis with a Parker plot using Vector NTI® Suite (Informax, Inc, Bethesda, Md.).

Any suitable method of immunization can be used. Such methods can include use of adjuvants, other immunostimulants, repeated booster immunizations, and the use of one or more immunization routes. Immunization can also be performed by DNA vector immunization, see, e.g., Wang, et al. (1997) Virology 228:278-284. Alternatively, animals can be immunized with cells bearing the antigen of interest, which may provide superior antibody generation than immunization with purified antigen (Kaithamana, et al. (1999) J. Immunol. 163:5157-5164).

Preferred antibody antagonists are monoclonal antibodies, which may be obtained by a variety of techniques familiar to skilled artisans. Methods for generating monoclonal antibodies are generally described in Stites, et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Typically, splenocytes isolated from an immunized mammalian host are immortalized, commonly by fusion with a myeloma cell to produce a hybridoma. See Kohler and Milstein (1976) Eur. J. Immunol. 6:511-519; Meyaard, et al. (1997) Immunity 7:283-290; Wright, et al. (2000) Immunity 13:233-242; Preston, et al. (1997) Eur. J. Immunol. 27:1911-1918. Alternative methods of immortalization include transformation with Epstein Barr Virus, oncogenes, or retroviruses, or other methods known in the art. See, e.g., Doyle, et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity, affinity and inhibiting activity using suitable binding and biological assays. For example, antibody to target binding properties can be measured, e.g., by surface plasmon resonance (Karlsson, et al. (1991) J. Immunol. Methods 145:229-240; Neri, et al. (1997) Nat. Biotechnol. 15:1271-1275; Jonsson, et al. (1991) Biotechniques 11:620-627) or by competition ELISA (Friguet, et al. (1985) J. Immunol. Methods 77:305-319; Hubble (1997) Immunol. Today 18:305-306).

Alternatively, one may isolate DNA sequences which encode a monoclonal antibody or a binding fragment thereof by screening a DNA library from human B cells, see e.g., Huse, et al. (1989) Science 246:1275-1281. Other suitable techniques involve screening phage antibody display libraries. See, e.g., Huse et al., Science 246:1275-1281 (1989); and Ward et al., Nature 341:544-546 (1989); Clackson et al. (1991) Nature 352: 624-628 and Marks et al. (1991) J. Mol. Biol. 222: 581-597; Presta (2005) J. Allergy Clin. Immunol. 116:731.

Preferred monoclonal antibodies for use in the present invention are “chimeric” antibodies (immunoglobulins) in which the variable domain is from the parental antibody generated in an experimental mammalian animal, such as a rat or mouse, and the constant domains are obtained from a human antibody, so that the resulting chimeric antibody will be less likely to elicit an adverse immune response in a human subject than the parental mammalian antibody. More preferably, a monoclonal antibody used in the present invention is a “humanized antibody”, in which all or substantially all of the hypervariable loops (e.g., the complementarity determining regions or CDRs) in the variable domains correspond to those of a non-human immunoglobulin, and all or substantially all of the framework (FR) regions in the variable domains are those of a human immunoglobulin sequence. A particularly preferred monoclonal antibody for use in the present invention is a “fully human antibody”, e.g., an antibody that comprises human immunoglobulin protein sequences only. A fully human antibody may contain carbohydrate chains from the cell species in which it is produced, e.g., if produced in a mouse, in a mouse cell, or in a hybridoma derived from a mouse cell, a fully human antibody will typically contain murine carbohydrate chains.

Monoclonal antibodies used in the present invention may also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079.

The antagonistic antibodies used in the present invention may have modified (or blocked) Fc regions to provide altered effector functions. See, e.g., U.S. Pat. No. 5,624,821; WO2003/086310; WO2005/120571; WO2006/0057702. Alterations of the Fc region include amino acid changes (substitutions, deletions and insertions), glycosylation or deglycosylation, and adding multiple Fc. Changes to the Fc can alter the half-life of therapeutic antibodies, enabling less frequent dosing and thus increased convenience and decreased use of material. See Presta (2005) J. Allergy Clin. Immunol. 116:731 at 734-35.

The antibodies may also be conjugated (e.g., covalently linked) to molecules that improve stability of the antibody during storage or increase the half-life of the antibody in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g., Chapman, A. P. (2002) Adv. Drug Deliv. Rev. 54:531-545; Anderson and Tomasi (1988) J. Immunol. Methods 109:37-42; Suzuki, et al. (1984) Biochim. Biophys. Acta 788:248-255; and Brekke and Sandlie (2003) Nature Rev. 2:52-62).

Bispecific antibodies that antagonize both IL-17 and IL-23 activity can be produced by any technique known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan, et al. (1985) Science 229: 81. These bifunctional antibodies can also be prepared by disulfide exchange, production of hybrid-hybridomas (quadromas), by transcription and translation to produce a single polypeptide chain embodying a bispecific antibody, or transcription and translation to produce more than one polypeptide chain that can associate covalently to produce a bispecific antibody. The contemplated bispecific antibody can also be made entirely by chemical synthesis. The bispecific antibody may comprise two different variable regions, two different constant regions, a variable region and a constant region, or other variations.

Antibodies used in the present invention will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

IL-17A antagonists and IL-23 antagonists are typically administered to a patient as pharmaceutical compositions in which the antagonist is admixed with a pharmaceutically acceptable carrier or excipient, see, e.g., Remington's Pharmaceutical Sciences and U.S. Pharmacopeia: National Formulary, Mack Publishing Company, Easton, Pa. (1984). The pharmaceutical composition may be formulated in any manner suitable for the intended route of administration. Examples of pharmaceutical formulations include lyophilized powders, slurries, aqueous solutions, suspensions and sustained release formulations (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The route of administration will depend on the properties of the antagonist or other therapeutic agent used in the pharmaceutical composition. Preferably, pharmaceutical compositions containing IL-17A antagonists and IL-23 antagonists are administered systemically by oral ingestion, injection or infusion by intravenous, intraperitoneal, intracerebral, intramuscular, intraocular, intraarterial, intracerebrospinal, intralesional, or pulmonary routes, or by sustained release systems such as implants. Injection of gene transfer vectors into the central nervous system has also been described (see, e.g., Cua, et al. (2001) J. Immunol. 166:602-608; Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350,466 and 6,316,024).

The pharmaceutical compositions used in the invention may be administered according to any treatment regimen that ameliorates or prevents joint destruction. Selecting the treatment regimen will depend on several composition-dependent and patient-dependent factors, including but not limited to the half-life of the antagonist, the severity of the patient's symptoms, and the type or length of any adverse effects. Preferably, an administration regimen maximizes the amount of therapeutic agent delivered to the patient consistent with an acceptable level of side effects. Guidance in selecting appropriate doses of therapeutic antibodies and small molecules is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Biological antagonists such as antibodies may be provided by continuous infusion, or by doses at intervals of, e.g., once per day, once per week, or 2 to 7 times per week, once every other week, or once per month. A total weekly dose for an antibody is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. 1 Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg basis. Determination of the appropriate dose is made by the clinician, e.g., using parameters or factors known or suspected in the art to affect treatment or predicted to affect treatment. Generally, the beginning dose is an amount somewhat less than the optimum dose and the dose is increased by small increments thereafter until the desired or optimum effect is achieved relative to any negative side effects.

In one preferred embodiment, the IL-17A antagonist is a humanized monoclonal antibody which comprises a light chain having SEQ ID NO:1 and a heavy chain having SEQ ID NO:2. This humanized Mab is preferably administered subcutaneously twice a week, weekly, biweekly, or monthly at a dose of between 10 mg and 2,000 mg, more preferably once or twice monthly between 20 mg and 400 mg, and even more preferably once monthly at a dose of between 40 mg and 100 mg. In one preferred embodiment, this humanized Mab is administered according to a dosage regimen that achieves serum concentrations of 0.1-100 μg/mL, or more preferably 1-10 μg/mL.

Treatment regimens using IL-17A antagonists will typically be determined by the treating physician and will take into account the patient's age, medical history, disease symptoms, and tolerance for different types of medications and dosing regimens. Generally the treatment regimen is designed to suppress the overly aggressive immune system, allowing the body to eventually re-regulate itself, with the result often being that after the patient has been kept on systemic medications to suppress the inappropriate immune response for a finite length of time (for example, one year), medication can then be tapered and stopped without recurrence of the autoimmune attack. Sometimes resumption of the attack does occur, in which case the patient must be re-treated.

Thus, in some cases, the physician may prescribe the patient a certain number of doses of the IL-17A antagonist to be taken over a prescribed time period, after which therapy with the antagonist is discontinued. Preferably, after an initial treatment period in which one or more of the acute symptoms of the disease disappear, the physician will continue the antagonist therapy for some period of time, in which the amount and/or frequency of antagonist administered is gradually reduced before treatment is stopped.

The present invention also contemplates treatment regimens in which an IL-17A antagonist is used in combination with an IL-23 antagonist. Such regimens may be especially useful in treating the acute phase of the inflammatory joint disease, in which the IL-17A antagonist inhibits the activity of existing Th₁₇ cells, while the IL-23 antagonist prevents the generation of new Th₁₇ cells. Such combination therapy may provide effective treatment using a lower dose of the IL-17A antagonist and/or administering the IL-17A antagonist for a shorter period of time. As symptoms ameliorate, therapy with the IL-17A antagonist is preferably discontinued, while administration of the IL-23 antagonist is continued to prevent generation of new autoreactive Th₁₇ cells that could lead to recurrence of the disease. The two antagonists may be administered at the same time in a single composition, or in separate compositions. Alternately, the two antagonists may be administered at separate intervals. Different doses of the antagonists may also be used. Similarly, an anti-IL-17A/IL-23 bispecific antibody may also be administered during the acute phase and gradually withdrawn, followed by treatment with anti-IL-23 antibody to maintain repression of the disease.

The treatment regimen may also include use of other anti-rheumatic drugs or other therapeutic agents, to ameliorate one or more symptoms of the inflammatory joint disease or to prevent or ameliorate adverse effects from the antagonist therapy. Examples of therapeutic agents that have been used to treat symptoms of inflammatory joint diseases are NSAIDs and DMARDs.

In any of the therapies described herein in which two or more different therapeutic substances are used (e.g., an IL-17A antagonist and an IL-23 antagonist, or an IL-17A antagonist and a different anti-rheumatic drug), it will be understood that the different therapeutic substances are administered in association with each other, that is, they may be administered concurrently in the same pharmaceutical composition or as separate compositions or the substances may be administered at separate times, and in different orders.

The effectiveness of the IL-17A antagonist therapy for inhibiting joint destruction in a particular patient can be determined using diagnostic measures such as reduction or occurrence of inflammatory symptoms (e.g., swollen and tender joint counts), patient assessment of pain; patient and evaluator global assessment of disease activity and other peripheral manifestations of underlying joint pathology. Diagnostic measurements of a subject to be treated or treated according to the invention can be compared to data obtained from a control subject or control sample, which can be provided as a predetermined value, e.g., acquired from a statistically appropriate group of control subjects.

### Example 1

### NHDF Assay for Anti-IL-17A Antibodies

The ability of anti-IL-17A antibodies useful in the present invention to block the biological activity of IL-17A is measured by monitoring rhIL-17A-induced expression of IL-6 in a normal human (adult) dermal fibroblast (NHDF) primary cell line. Briefly, various concentrations of an anti-IL-17A antibody to be assayed are incubated with rhIL-17A, and the resulting mixture is then added to cultures of NHDF cells. IL-6 production is determined thereafter as a measure of the ability of the antibody in question to inhibit IL-17A activity. A more detailed protocol follows.

A series two-fold dilutions of anti-IL-17A antibodies of interest are prepared (in duplicate) starting with a stock solution at 40 μg/ml. A stock solution of rhIL-17A is prepared at 120 ng/ml. Seventy μl of the rhIL-17A stock solution is mixed with 70 μl of the anti-IL-17A antibody dilutions in wells of a microtiter plate and incubated at room temperature for 20 minutes. One hundred μl of each of these mixtures is then added to wells of a microtiter plate that had been seeded with 1×10⁴ NHDF cells/well (100 μl) the previous night and allowed to incubate at 37° C. NHDF cells (passage 4) were obtained from Cambrex BioScience (Baltimore, Md., USA). The resulting final concentration of rhIL-17A is 30 ng/ml (1 nM), and the antibodies range downward in two-fold intervals from 10 μg/ml. Plates are incubated at 37° C. for 24 hours, followed by harvesting of the supernatant and removal of 50 μl for use in an IL-6 ELISA.

The ELISA for detection of human IL-6 is performed as follows. Reagents are generally from R&D Systems (Minneapolis, Minn., USA). An hIL-6 capture antibody (50 μl/well of a 4 μg/ml solution) is transferred to wells of a microtiter plate, which is sealed and incubated overnight at 4° C. The plate is washed three times, and then blocked with 100 μl/well of blocking buffer for 1 hour or more. The plate is then washed again three times. Experimental samples (50 μl of the culture supernatant) and controls (serial dilutions of IL-6 protein) are added to the wells in 50 μl and incubated for two hours. Plates are washed three times, and 50 μl/well of a biotinylated anti-IL-6 detection antibody (300 ng/ml) is added. The plates are incubated at room temperature for two hours, washed three times, and 100 μl/well of streptavidin HRP is added and incubated for 20 minutes. The plate is washed again, ABTS (BioSource, Carlsbad, Calif., USA) is added (100 μl/well), and incubated for 20 minutes. Stop solution is added (100 μl/well) and the absorbance at 405 nm is measured.

The IC50 for an anti-IL-17A antibody of interest is the concentration of antibody required to reduce the level of rhIL-17A-induced IL-6 production to 50% of the level observed in the absence of any added anti-IL-17A antibody.

### Example 2

### Foreskin Fibroblast Assay Anti-IL-17A Antibodies

The ability of anti-IL-17A antibodies useful in the present invention to block the biological activity of IL-17A is measured by monitoring rhIL-17A-induced expression of IL-6 in HS68 foreskin fibroblast cell line. Reduced production of IL-6 in response to rhIL-17A is used as a measure of blocking activity by anti-IL-17A antibodies useful in the present invention.

Analysis of the expression of IL-17RC (an IL-17A receptor) in a panel of fibroblast cell lines identified the human foreskin fibroblast cell line HS68 (ATCC CRL1635) as a potential IL-17A responsive cell line. This was confirmed by indirect immunofluorescence staining with polyclonal goat anti-human IL-17R antibody (R&D Systems, Gaithersburg, Md., USA) followed by phycoerythrin (PE)-F(ab′)₂ donkey anti-goat IgG (Jackson Immunoresearch, Inc., West Grove, Pa., USA), and analyzing the PE immunofluorescence signal on a flow cytometer (FACScan, Becton-Dickinson, Franklin Lakes, N.J., USA). As further validation of the model, IL-17A (both adenovirus-derived rhIL-17A and commercially available E. coli-derived IL-17A, R&D Systems) induced a dose-responsive induction of IL-6 in the HS68 cells with an EC50 of 5-10 ng/ml, which induction was blocked by pre-incubation with commercial polyclonal and monoclonal anti-IL-17A antibodies (R&D Systems).

The IL-17A inhibition assay is performed as follows. A confluent T-75 flask of HS68 cells (approximately 2×10⁶ cells) is washed with Dulbecco's PBS without Ca++ and Mg++ and then incubated with 5 ml of cell dissociation medium (Sigma-Aldrich, St. Louis, Mo., USA) for 2-5 minutes at 37° C. in an incubator at 5% CO₂. Cells are then harvested with 5 ml of tissue culture (TC) medium and centrifuged for 5 minutes at 1000 rpm. TC medium is Dulbecco's Modified Eagle's Medium (with glutamine), 10% heat-inactivated fetal bovine serum (Hyclone), 10 mM Hepes, 1 mM sodium pyruvate, penicillin, and streptomycin. Cells are resuspended in 2 ml TC medium, diluted 1:1 with trypan blue and counted. Cell concentrations are adjusted to 1×10⁵ cells/ml in TC medium, and 0.1 ml/well is aliquoted into the wells of a flat-bottom plate containing 0.1 ml TC medium. Cells are grown overnight and the supernatant is aspirated and cells are washed with 0.2 ml of fresh TC medium.

Anti-IL-17A antibodies to be assayed are serially diluted in two-fold or 3-fold steps to give a series of stock solutions that can be used to create final antibody concentrations of 1 to 0.001 μg/ml in the IL-17A inhibition assay. A rat IgG control is used in each assay, as well as media-only samples, as controls to measure spontaneous IL-6 production in HS68 cells. The TC medium is aspirated from the wells of the plate containing the HS68 cells. Aliquots of the various concentrations of anti-IL-17A antibody (0.1 ml of each) are pre-incubated in the wells with the HS68 cells 37° C. for 5 minutes prior to addition of 0.1 ml of 20 ng/ml rhIL-17A, to give a final concentration of rhIL-17A of 10 ng/ml (approximately 330 μM of IL-17A dimer). Cells are incubated 24 hours at 37° C., and supernatants (50-100 μl) are harvested and assayed for IL-6, for example using a human IL-6 ELISA kit from Pharmingen (OptEIA-BD Biosciences, Franklin Lakes, N.J., USA).

### Example 3

### Ba/F3-hIL-17Rc-mGCSFR Proliferation Assay

The ability of the anti-IL-17A antibodies useful in the present invention to block the biological activity of IL-17A is measured by monitoring rhIL-17A-induced proliferation of a cell line engineered to proliferate in response to IL-17A stimulation. Specifically, the Ba/F3 cell line (IL-3 dependent murine pro-B cells) was modified to express a fusion protein comprising the extracellular domain of a human IL-17A receptor (hIL-17RC) fused to the transmembrane domain and cytoplasmic region of mouse granulocyte colony-stimulating factor receptor (GCSFR). The resulting cell line is referred to herein as Ba/F3 hIL-17Rc-mGCSFR. Binding of homodimeric IL-17A to the extracellular IL-17RC domains causes dimerization of the hIL-17Rc-mGCFR fusion protein receptor, which signals proliferation of the Ba/F3 cells via their mGCSFR cytoplasmic domains. Such cells proliferate in response to IL-17A, providing a convenient assay for IL-17A antagonists, such as anti-IL-17A antibodies.

The sensitivity of the Ba/F3-hIL-17Rc-mGCSFR proliferation assay to IL-17A stimulation makes it possible to perform experiments at relatively low concentrations of rhIL-17A (e.g. 3 ng/ml, 100 pM) compared with other assays, while still maintaining a robust and readily measurable proliferative response. This means that lower concentrations of anti-IL-17A antibodies are required to achieve a molar excess over rhIL-17A in the assay. Experiments performed at lower antibody concentrations make it possible to discriminate between high affinity antibodies that might otherwise be indistinguishable (i.e. experiments can be performed closer to the linear range in the antibody-IL-17A binding curve, rather than in the plateau).

### Example 4

### Treatment of Collagen-Induced Arthritis Using Anti-IL-17A Antibodies

Collagen-induced arthritis (CIA) is a widely accepted mouse model for rheumatoid arthritis in humans. Rat anti-IL-17A antibody JL7.1D10, which binds to mouse IL-17A with high affinity, was administered to mice expressing CIA to assess the ability of anti-IL-17A therapy to treat rheumatoid arthritis.

The procedure was as follows. On Day 0 male B10.RIII mice were immunized intradermally at the tail base with bovine type II collagen emulsified in Complete Freund's Adjuvant. On Day 21 mice were challenged intradermally with bovine type II collagen emulsified in Incomplete Freund's Adjuvant delivered at the tail base. When the first sign of severe arthritis in the immunized group occurred (post-Day 21), all remaining immunized mice were randomized to the various treatment groups. Animals were treated with either 800 μg, 200 μg, or 50 μg of anti-IL-17A antibody JL7.1D10; 200 μg isotype control antibody; or diluent. JL7.1D10 is a surrogate, neutralizing, very high affinity rat antibody specific for mouse IL-17A (and human IL-17A) (hereinafter 1D10). Treatments were given subcutaneously on the first day of disease onset in the immunized mice, and then weekly four more times. Mice were sacrificed at day 35 and paws were fixed in 10% neutral-buffered formalin for tissue processing and sectioning. Paws were analyzed by a pathologist for the following histopathology parameters: reactive synovium, inflammation, pannus formation, cartilage destruction, bone erosion, and bone formation. Each parameter was graded using the following disease scale: 0=no disease; 1=minimal, 2=mild, 3=moderate, 4=severe. In addition paws were assessed using visual disease severity score (DSS), which measures swelling and redness on a scale of 0 to 3, with 0 being a normal paw, 1 being inflammation of one finger on the paw, 2 being inflammation of two fingers or the palm of that paw, and 3 being inflammation of the palm and finger(s) of the paw. Scores of 2 and 3 are referred to herein as severely or highly inflamed paws.

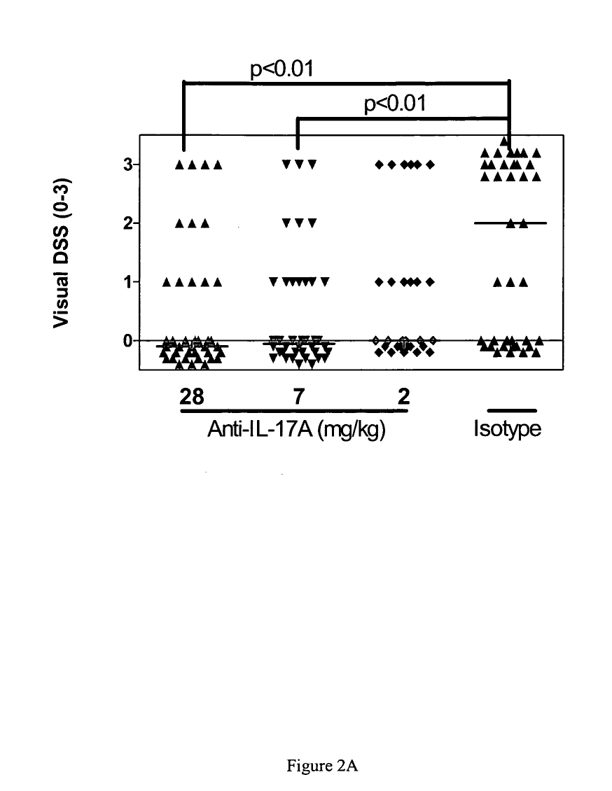

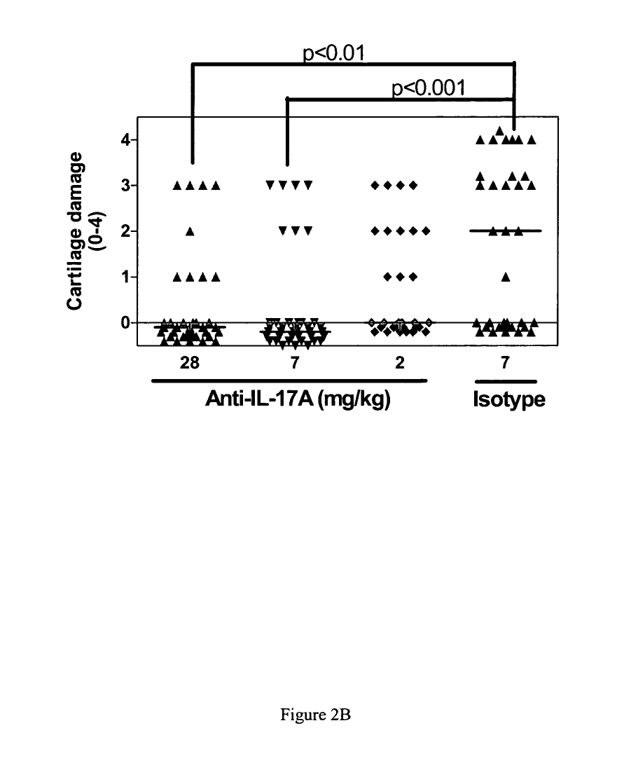

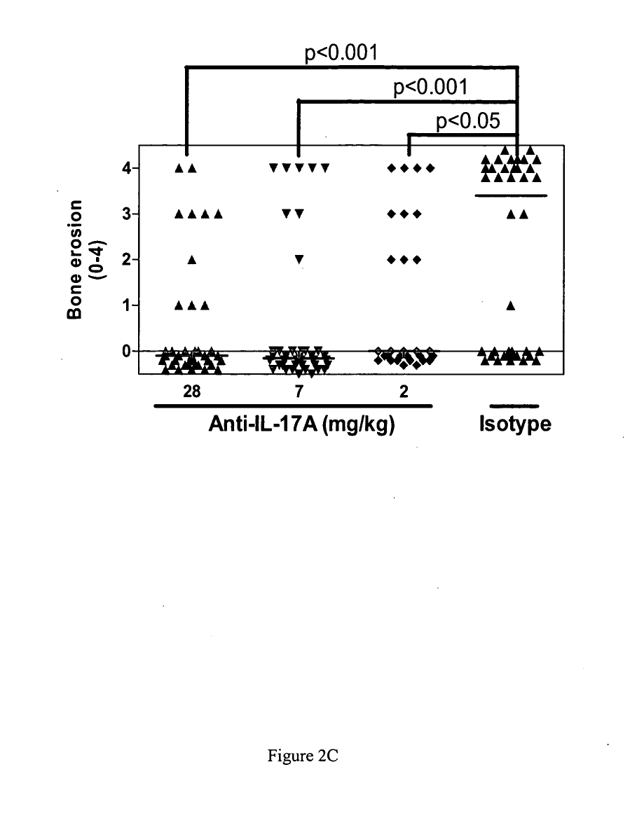

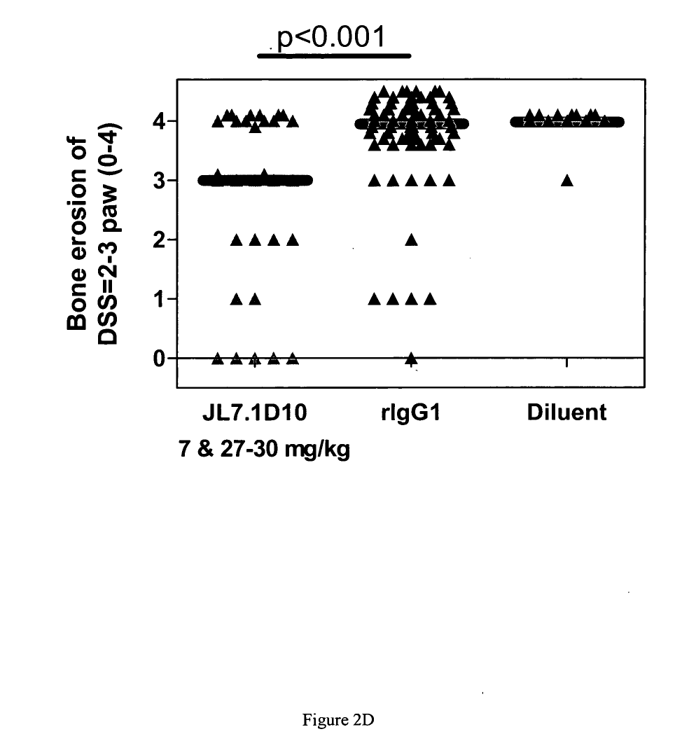

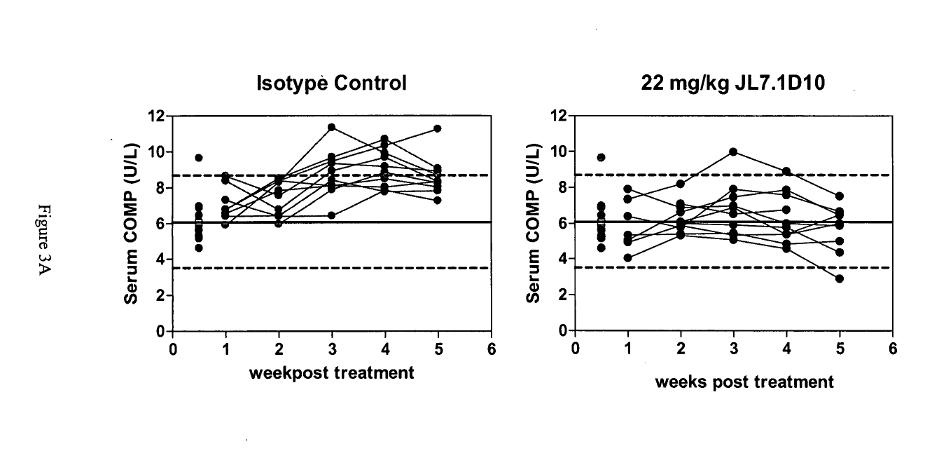

Results are presented at FIGS. 2A-2C. Each data point represents one paw, rather than an average for all four paws for an animal or an average over all animals. Reduction in the number of paws showing high pathology scores was statistically significant by three measures of pathology (visual DSS—paw swelling and redness, cartilage damage and bone erosion) with higher anti-IL-17A 1D10 concentrations tested (28 and 7 mg/kg). Results with the lowest concentration (2 mg/kg) were statistically significant for bone erosion and reduced for visual DSS and cartilage damage. Similar benefits were observed in reduction of production of cartilage degradative enzymes within inflamed paws (matrix metalloproteases MMP-2, MMP-3, MMP-13).

Visual evaluation of paw inflammation, however, may underestimate the therapeutic benefit of anti-IL-17A treatment of CIA mice, e.g. decreased bone erosion. In other experiments, highly inflamed paws (DSS scores of 2 or 3) from CIA mice were analyzed for bone erosion using histopathology or micro-computed tomography (micro-CT). This study was possible because even though anti-IL-17A-treated animals had drastically reduced percentages of highly inflamed paws (see, e.g., FIG. 2A), there remained a number of highly inflamed paws, and it was possible to compare highly inflamed paws (DSS=2 or 3) from all treatment groups, including the no-antibody controls. FIG. 2D shows a plot of bone erosion for highly inflamed paws from diluent treated, isotype control (rIgG1) treated, and anti-IL-17A antibody treated animals. Bone erosion, as measured by histopathology, was significantly reduced in paws from animals treated with anti-IL-17A when compared with no-antibody controls, despite their similar DSS scores. The results suggest that sparing of bone erosion may be achieved with anti-IL-17A treatment even in paws where there is no apparent improvement in inflammation as measured by DSS score.

Similar results were obtained when micro-CT was used to measure bone mineral density (BMD) for joints in highly inflamed paws in CIA mice. Table 1 provides BMD for paws with disease severity scores of 0 or 3 from CIA animals treated with either anti-IL-17A antibody 1D10 or an isotype control (25D2). Even for joints with the same visual disease severity, 1D10 antibody treated mice had only approximately half the decrease in bone mineral density observed with isotype control treated animals.

TABLE 1
Bone Density for Joints in CIA Mice

| Treatment | DSS | BMD (mg/cc) |
|---|---|---|
| 25D2 | 3 | 95 |
| 25D2 | 3 | 108 |
| 1D10 | 3 | 288 |
| 1D10 | 3 | 299 |
| 1D10 | 0 | 502 |
| 1D10 | 0 | 480 |

As with bone erosion, cartilage destruction and pannus formation (proliferation of the synovial lining forming excessive folds of inflamed tissue) were also reduced in 1D10-treated CIA mice. Histopathology showed that anti-IL-17A antibody treatment not only reduced the number of paws showing severe pathology, but also reduced pathology in paws that appeared equally inflamed based on visual inspection (DSS scores of 2 and 3) when compared with diluent or isotype treated controls.

The observation that treatment with an anti-IL-17A antibody significantly reduced bone erosion in the CIA model of joint inflammation suggests that such therapy may be useful in preventing one of the most debilitating and irreversible effects of RA in humans. In addition, the observation that bone erosion is reduced even in highly inflamed paws suggests that simple visual assessment of joint inflammation may not accurately measure therapeutic efficacy. Direct or indirect measurement of bone erosion may be necessary to track the effects of therapeutic treatments. Direct methods include, but are not limited to, standard 2-D X-ray detection, computed tomography (CT), magnetic resonance imaging (MRI), ultrasound (US), and scintigraphy. See, e.g., Guermazi et al. (2004) Semin. Musculoskelet. Radiol. 8(4):269-285. Indirect methods include the joint destruction biomarkers described herein.

### Example 5

### Modulation of Serum COMP Levels in CIA Mice by Anti-IL-17A Therapy

The CIA mice model of arthritis was used to assess the effect of antibody 1D10 on serum levels of COMP, which is a non-collagenous protein incorporated into the cartilage matrix and is released into the synovial fluid and serum following cartilage proteolysis. Synovial fluid and serum COMP can also arise via de novo synthesis (i.e. un-related to cartilage destruction).

B10.RIII mice were immunized and boosted with type II collagen. After the first mice in the cohort of immunized/boosted mice developed a severely inflamed paw, all mice were randomized to receive therapy. Isotype or rat anti-mouse antibody 1D10 were delivered SC weekly for 5 weeks. Mice were bleed prior to the 2^{nd}, 3^{rd}, 4^{th}, and 5^{th} dose and then at sacrifice. Un-manipulated mice were bled to determine the normal range of serum COMP in the absence of disease. Serum COMP levels in CIA mice were measured using a commercial animal COMP ELISA (MD Biosciences, St. Paul, Minn.).

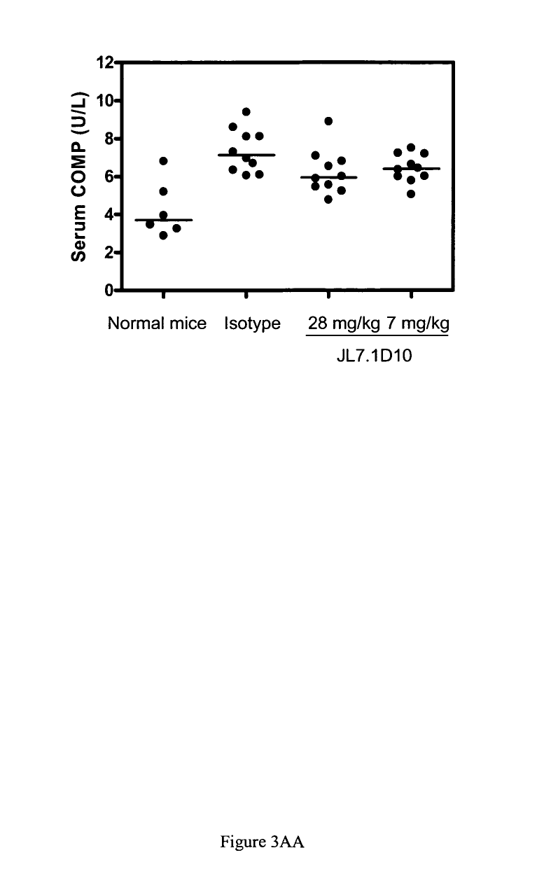

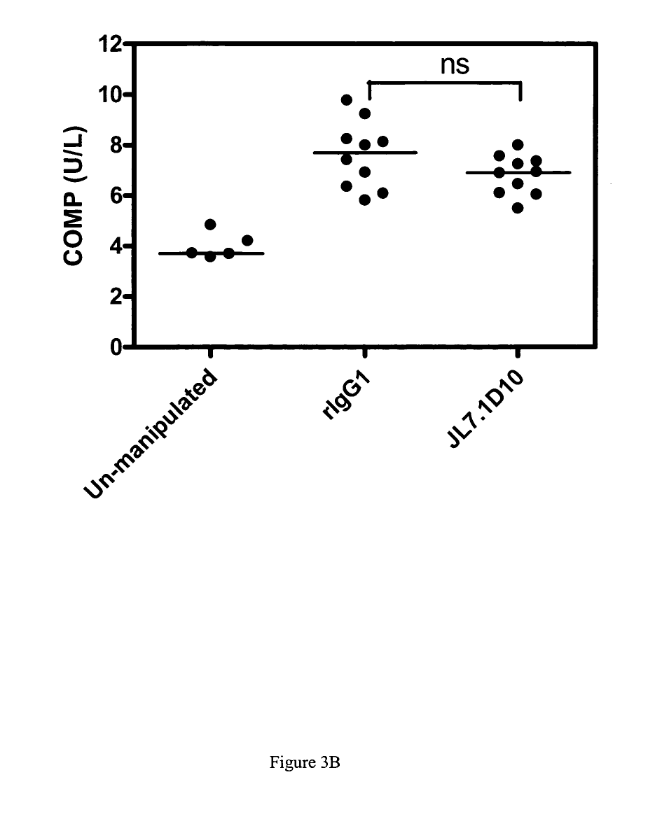

The results are shown in FIG. 3A: the solid horizontal bar denotes the average serum COMP in non-diseased animals (grey circles on left of graphs) and the two dotted horizontal bars denotes +/−2 standard deviations from the non-diseased mice average. Note the one outlier non-diseased mouse that significantly pulls the dotted horizontal bars away from the average.

The results from this experiment show that arthritic mice have elevated serum COMP levels compared to the range of serum COMP levels seen in non-arthritic mice and that exposure to an anti-IL-17A antibody decreased the elevated serum COMP levels in the mice primed to get CIA. These decreased COMP levels correlated with reduced cartilage destruction as evidenced by joint histology data obtained from the same experiment using standard histological methods (data not shown).

To evaluate the dose-dependence of anti-IL-17A therapy on serum COMP levels, a second experiment was performed as described above, except that the weekly dose of anti-IL-17A antibody 1D10 was 28 mg/kg, 7 mg/kg or 2 mg/kg. No change in COMP levels were observed with the 2 mg/kg dose; however, a trend towards decreased COMP levels was observed in the CIA mice treated with either of the two higher doses, as shown in FIG. 3AA.

The effect of short term anti-IL-17A therapy on serum COMP levels was investigated in severely arthritic mice. B10.RIII mice were immunized and boosted with type II collagen. Each mouse was examined and treated with a single dose of isotype (rat IgG1) or antibody 1D10 (28 mg/kg) when it exhibited a severely inflamed paw. Mice were sacrificed seven days after antibody treatment. Un-manipulated mice were bled to determine the normal range of serum COMP in the absence of disease. The results are shown in FIG. 3B. 1D10 treated mice trended to reduced serum COMP after 7 days of drug exposure; therefore, longer drug exposure may be needed to bring COMP levels down to un-manipulated levels.

The results of these experiments on mice primed to get CIA and severely arthritic mice are consistent with the ability of 1D10 to inhibit cartilage destruction as measured by standard histological methods. Thus, the cartilage preserving properties of IL-17A antagonism correlated with this serum marker of cartilage destruction and serum COMP is likely to be useful as a biomarker of the effect of anti-IL-17A therapy on joint destruction in human RA patients.

### Example 6

### Modulation of Serum RANKL Levels in CIA Mice by Anti-IL-17A Therapy

The CIA mice model of arthritis was used to assess the effect of anti-IL-17A therapy on serum levels of RANKL, which is a cell-surface molecule expressed by activated T-cells, synoviocytes, and osteoblasts that regulates the developmental transition of pre-osteoclasts into mature osteoclasts. Serum RANKL is elevated in human RA.

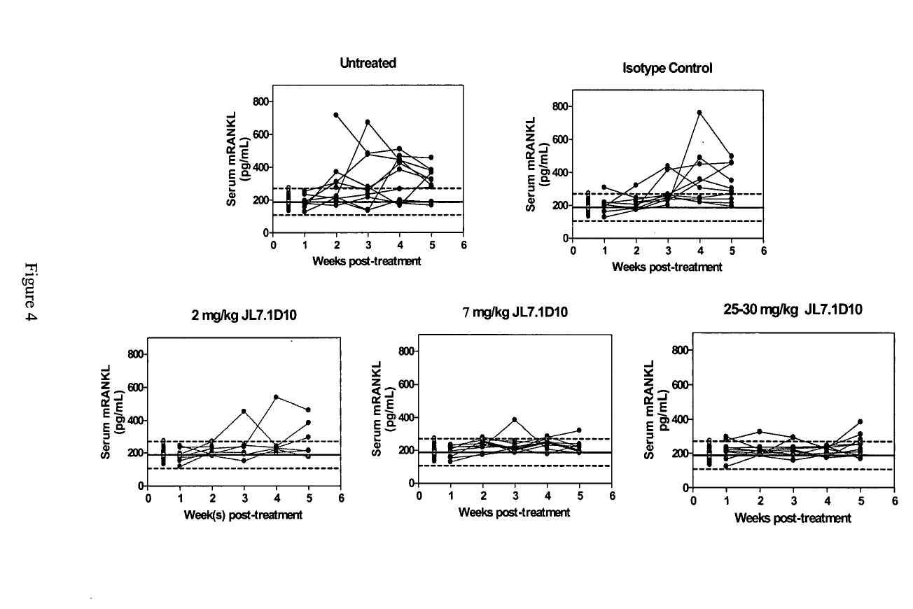

B10.RIII mice were immunized and boosted with type II collagen. After the first mice in the cohort of immunized/boosted mice developed a severely inflamed paw, all mice were randomized to receive therapy. Vehicle, isotype antibody, or one of three different doses of antibody 1D10 were delivered SC weekly for 5 weeks. Mice were bleed prior to the 2^{nd}, 3^{rd}, 4^{th}, and 5^{th} dose and then at sacrifice. Un-manipulated mice were bled to determine the normal range of serum RANKL in the absence of disease. The results are shown in FIG. 4: the solid horizontal bar denotes the average serum RANKL level in non-diseased animals (grey circles on left of graphs) and the two dotted horizontal bars denotes +/−2 standard deviations from the non-diseased mice average.

Non-diseased mice (solid bar, grey circles) have detectable serum RANKL levels, but arthritic mice have elevated levels (vehicle). Exposure to antibody 1D10 decreased the elevated serum RANKL levels in the mice primed to get CIA compared to isotype (Rat IgG1), with the highest dose (28 mg/kg) bringing the serum RANKL level for each animal within the normal range. Significantly, this dose of 1D10 is also effective at inhibiting bone erosion as measured by standard histological methods. Thus, since the bone preserving properties of IL-17A antagonism correlated with this serum marker of bone destruction, RANKL is likely to be useful as a biomarker of the effect of anti-IL-17A therapy on joint destruction in human RA patients. These results also suggest that endogenous IL-17A plays a major role in RANKL production in CIA mice, and that inhibition of RANKL expression by the anti-IL-17A 1D10 antibody may partially explain why this antibody inhibited joint bone erosion, even in the few paws that becamse severely swollen.

### Example 7

### Modulation of Serum OPG Levels in CIA Mice by Anti-IL-17A Therapy

The CIA mice model of arthritis was used to assess the effect of anti-IL-17A therapy on serum levels of OPG, which is a soluble factor that binds to cell-surface RANKL and soluble RANKL and antagonizes RANKL from delivering the developmental signal to pre-osteoclasts. OPG is elevated in human RA serum, as is RANKL, which may reflect the organism's attempt to diminish the elevated RANKL's effects.

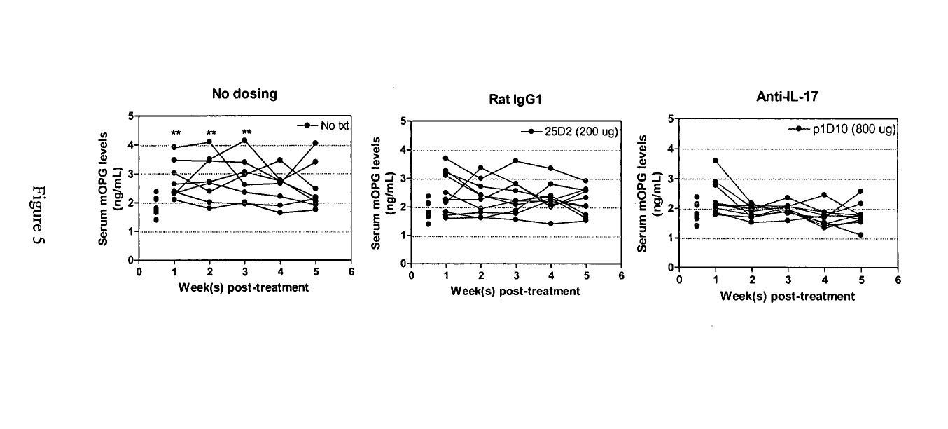

B10.RIII mice were immunized and boosted with type II collagen. After the first mice in the cohort of immunized/boosted mice developed a severely inflamed paw, all mice were randomized to receive therapy. Isotype or 1D10 were delivered SC weekly for 5 weeks. Mice were bleed prior to the 2^{nd}, 3^{rd}, 4^{th}, and 5^{th} dose and then at sacrifice. Un-manipulated mice were bled to determine the normal range of serum OPG in the absence of disease. The results are shown in FIG. 5.

Non-diseased mice have detectable serum OPG levels (grey circles on left of graphs), but arthritic mice had elevated levels (no dosing graph). Antibody 1D10 exposure decreased the elevated serum OPG levels in the mice primed to get CIA, whereas the isotype control did not. Thus, the bone preserving properties of IL-17A antagonism correlated with this serum marker of bone destruction and OPG is likely to be useful as a biomarker of the effect of anti-IL-17A therapy on joint destruction in human RA patients.

### Example 8

### Modulation of Serum RANKL Levels in CIA Mice by Short Term Anti-IL-17A Therapy

The short term effect of anti-IL-17A antibody 1D10 on serum RANKL and OPG in severely arthritic mice was also assessed.

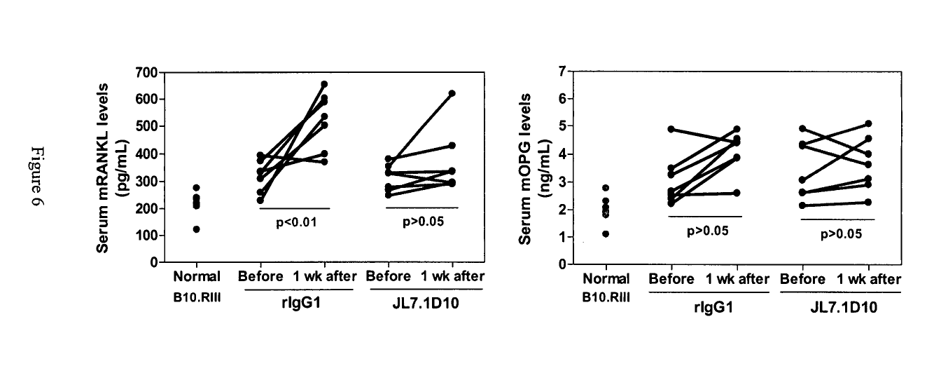

B10.RIII mice were immunized and boosted with type II collagen. Each mouse was examined and treated intravenously with a single 7 mg/kg dose of isotype or JL7.1D10 when it exhibited a severely inflamed paw (DSS≧2). Mice were sacrificed seven days after antibody treatment. Un-manipulated mice were bled to determine the normal range of serum RANKL and OPG in the absence of disease. Serum JL7.1D10 concentrations were quantified over the course of the experiment to confirm drug exposure throughout the experiment (data not shown). Statistical analysis was performed using a paired t test and a p value of 0.05 used to delineate significant differences between groups. The results are shown in FIG. 6.

Serum RANKL and OPG concentrations were already elevated in severely arthritic mice (mice with DSS≧2) at the time of antibody treatment. At day 7 post treatment, the elevation in serum RANKL levels observed in the isotype control group (p<0.01) was minimized by the 1D10 treatment (left panel). The same 1D10 exposure had no immediate impact on the elevated serum OPG (right panel), suggesting that the compensatory OPG decrease takes greater than 7 days of 1D10 exposure to trigger. These results further support the expectation that both RANKL and OPG could be surrogate biomarkers for efficacy of anti-IL-17A therapy in inhibiting joint destruction in inflammatory joint disease.

When the same experiment was performed using a subcutaneous (SC) route of delivery; significantly further elevated serum RANKL was observed with and without IL-17A neutralization. The discrepancy between the results of these experiments may be due to one or more of the following reasons: route of dosing, antibody batch, or variation in disease severity post-boost.

### Example 9

### Anti-IL-17A Inhibition of Joint Destruction in Inflammatory Nonresponders

The above examples demonstrate that IL-17A antagonism with antibody 1D10 decreased bone erosion even in a severely inflamed paw by histopathology and that elevated RANKL and OPG were normalized by 1D10 exposure. Paws from control antibody and 1D10 treated animals were submitted for micro-CT analysis as an additional method to evalute 1D10's effect on bone metabolism.

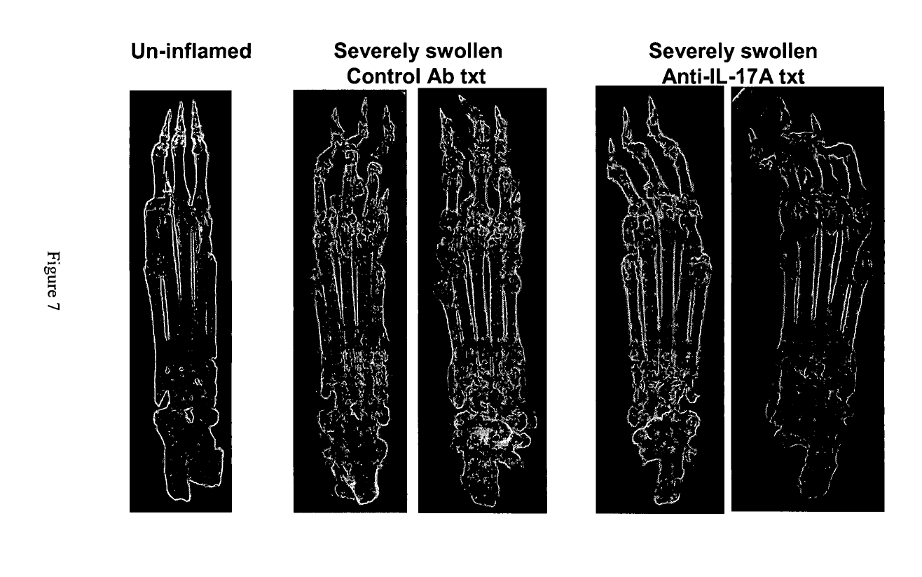

B10.RIII mice were immunized and boosted with type II collagen. After the first mice in the cohort of immunized/boosted mice developed a severely inflamed paw, all mice were randomized to receive therapy. The few severely inflamed paws from JL7.1D10 treated mice were compared with the more numerous severely inflamed paws from control antibody treated mice by micro-CT X-ray analysis and the results are shown in FIG. 7.

An un-inflamed paw has no evidence of bone erosion at the articular surface; however, a severely inflamed paw from a control treated mouse has extensive bone erosion at the articular surface where the immunogen type II collagen is present. This is evidenced by the replacement of very defined X-ray-dense bone structures at the ends of bones with amorphous X-ray-dense areas around the articular joints. The few severely inflamed joints from 1D10 treated mice have evidence of bone erosion, but the degree of bone erosion is much less that “equally inflamed” paws from control treated mice.

These micro-CT X-Ray results support the rationale that IL-17A antagonism in RA patients will result in reduced bone erosion in the joints versus placebo, even in patients that continue to have active disease as assessed by tender joint count and swollen joint counts. Or stated a different way, IL-17A antagonism can un-couple the outward signs of inflammation from the joint destructive process.

### Example 10

### Effect of Anti-IL-17A Therapy on Serum TRACP Levels in CIA Mice

The effect of long-term anti-IL-17A therapy on serum TRACP levels was also assessed.

B10.RIII mice were immunized and boosted with type II collagen. After the first mice in the cohort of immunized/boosted mice developed a severely inflamed paw, all mice were randomized to receive therapy. Isotype rat IgG1 or JL7.1D10 were dosed s.c. weekly for 5 weeks. Mice were bled at sacrifice. Un-manipulated naïve mice were bled to determine the normal range of serum TRACP in the absence of disease. Serum TRACP levels in CIA mice and un-manipulated mice were measured using a commercial mouse TRACP assay (IDS, Fountain Hills, Ariz.).

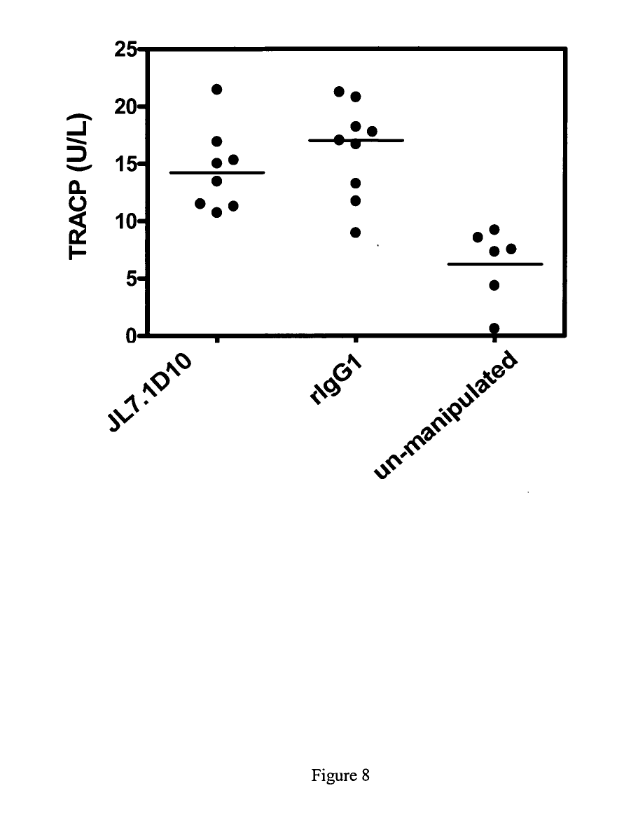

Serum TRACP levels were elevated in arthritic mice treated with the isotype rIgG1 control compared to non-diseased (un-manipulated) mice (FIG. 8), and these elevated TRACP levels correlated with the level of bone destruction in the swollen paws of the arthritic mice (data not shown). A slight trend to lower serum TRACP levels was observed in arthritic mice treated with 1D10, but this result was not statistically significant (FIG. 8). The inventors believe that the failure to see a statistically significant reduction in TRACP levels in this experiment may be due to the terminal sacrifice bleed time point at which TRACP levels were measured for the reasons discussed below.

### Example 11

### Effect of Anti-IL-17A Therapy on Serum CTX-1 and CTX-2 Levels in CIA Mice

B10.RIII mice were immunized and boosted with type II collagen. After the first mouse in the cohort of immunized/boosted mice developed a severely inflamed paw, all mice were randomized to receive therapy. Isotype or JL7.1D10 (28, 7 or 2 mg/kg) were delivered s.c. weekly for five weeks. Mice were bled at day 35 post treatment at sacrifice. Un-manipulated B10.RIII mice were bled to determine the normal range of CTX-1 in the absence of arthritis. Serum CTX-I levels were measured using the RatLaps™ CTX-I ELISA (IDS, Fountain Hills, Ariz.), which recognizes mouse CTX-I. CTX-II levels were measured using the serum pre-clinical CartiLaps® CTX-II ELISA (IDS, Fountain Hills, Ariz.). The results (not shown) indicate that (1) CTX-I was present in un-manipulated mouse serum, but was not elevated in arthritic mice serum, and (2) CTX-II was below the limit of detection in non-arthritic (normal) and arthritic mouse serum. JL7.1D10 did not detectably alter the basal levels for CTX-1 or CTX-H.

### Example 12

### Effect of Anti-IL-17A Therapy on Serum Osteocalcin Levels in CIA Mice

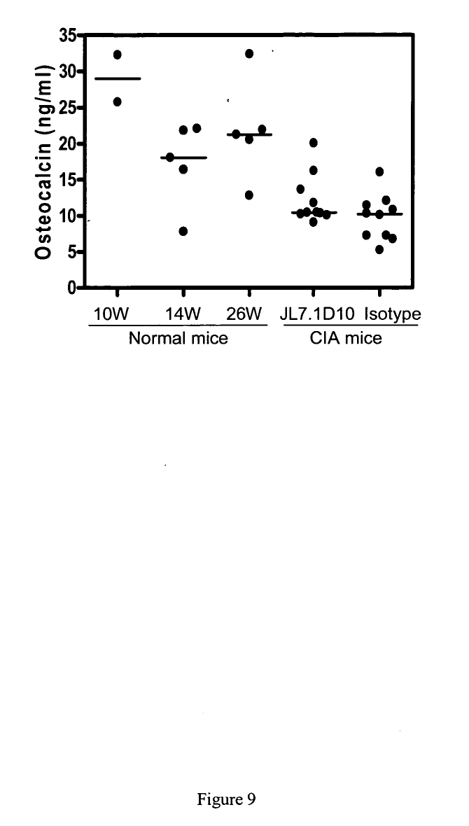

B10.RIII mice were immunized and boosted with type H collagen. After the first mouse in the cohort of immunized/boosted mice developed a severely inflamed paw, all mice were randomized to receive therapy. Isotype or 22 mg/kg of JL7.1D10 were delivered s.c. weekly for five weeks. Mice were bled at day 35 post treatment at sacrifice. Un-manipulated mice were bled at 10 weeks, 14 weeks, and 26 weeks of age to determine normal range of osteocalcin throughout arthritis model. Serum osteocalcin levels were measured using a commercial mouse osteocalcin sandwich ELISA assay (Biomedical Technologies Inc., Stoughton, Mass.). The results are shown in FIG. 9.

Arthritic mice treated with the isotype control had depressed osteocalcin levels compared to the levels in un-manipulated (normal) mice. While treatment with the JL7.1D10 antibody did not significantly modulate these levels, there was a trend to normalization of the depressed osteocalcin levels by JL7.1D10.

### Example 13

### Dynamics of Serum RANKL and OPG Levels as Mice Progress through the CIA Model

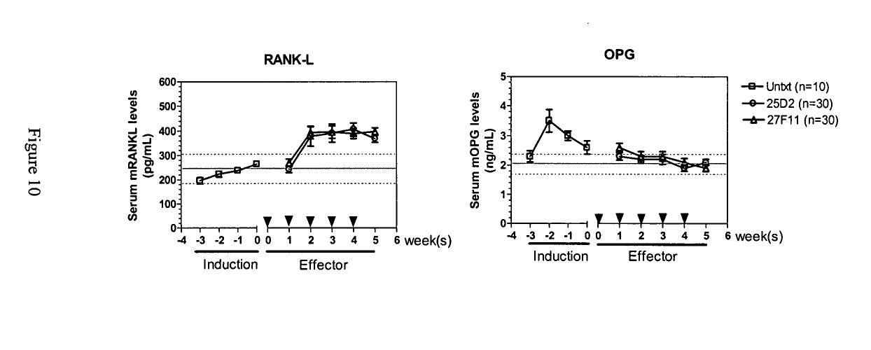

Male B10.RII mice were immunized with CII in CFA and bleed weekly for 3 weeks during the induction period (Untxt). Mice were then boosted with CII in WA and randomized to various treatment groups at the first sign of severe paw swelling. Some mice were dosed with 7-30 mg/kg control antibodies 25D2 or 27F11 subcutaneously weekly for 5 weeks. The results were pooled from five independent experiments and expressed as means ±SEM. of arthritic groups (n=10-30). The results are shown in FIG. 10.

As seen in the left panel of FIG. 10, serum RANKL levels increased starting the second week of the effector phase (disease progression) and remained elevated in the cohort of immunized/boosted mice for four weeks post-boost compared with un-manipulated control mice (horizontal lines). Elevated serum OPG levels, in contrast, appeared in the first week of the induction phase and were returning to baseline by the second week of the effector phase (FIG. 10, right panel). These results indicate that serum RANKL levels were elevated during the disease progression phase of the model when joint bone erosion occurs within severely swollen arthritic paws. RANKL's natural antagonist, OPG, which had been elevated during the induction phase was back to normal physiologic levels by the time that the pro-osteoclast RANKL was starting to rise in the serum. Serum OPG levels were 5-10 times higher than serum RANKL levels regardless of the time point examined. The increased serum RANKL is most likely complexed with the higher serum OPG levels and neutralized.

### Example 14

### Correlation of Elevated RANKL with Paw Swelling in CIA Mice

The data discussed in Example 13 shows that serum RANKL is elevated in the CIA model over a certain time-course and previous data showed that paw swelling occurred over a similar time course. To assess the degree of correlation between serum RANKL levels and paw swelling, mice were immunized, boosted, randomized to treatment at the first sign of severe paw swelling, and then treated with rat IgG1 isotype control antibody subcutaneously weekly for 5 weeks. Each week's serum RANKL concentration versus total animal DSS from individual mice was compiled from four independent experiments and the statistical analysis of the data was performed using non-parametric Kruskal-Wallis analysis.

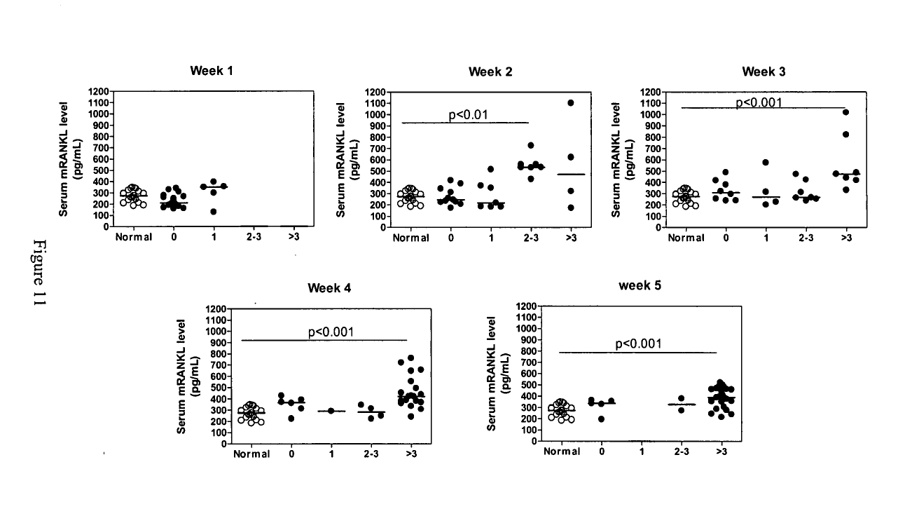

FIG. 11 shows the resulting week by week snapshot association between serum RANKL levels and disease activity in CIA mice treated with the isotope control antibody. Serum RANKL levels were only elevated in those mice that had at least one severely swollen paw (DSS=2-3) at week 2 or that had multiple severely swollen paws (DSS>3) from weeks 3-5. Importantly, mice immunized and boosted that had not initiated any paw swelling response (DSS=0) or that only initiated a mild paw swelling response in only one paw (DSS=1) showed no signs of elevated serum RANKL at any time point. A similar weekly profile between serum RANKL levels and disease activity in CIA mice was observed when mice were treated with a different control antibody (mouse IgG1 antibody 27F11). The data with both control antibodies support a conclusion that serum RANKL levels are elevated in CIA mice at week 2 and beyond once the mice demonstrate at least one severely swollen paw, and conversely was not just a reflection of time post-challenge, i.e. immunized/boosted mice with no signs of paw swelling did not have elevated serum RANKL.

Companion paw histology assessment studies concluded that significant bone erosion only occurred in severely swollen paws (DSS=2-3), not in minimally swollen paws (DSS=1) and not in un-inflamed paws from mice with “other paws” severely inflamed (DSS=0). Therefore, the inventors herein believe that elevated serum RANKL is directly correlated with the on-going bone erosion in the CIA model and that serum RANKL could be used as a PK-PD marker to follow response to experimental or approved therapies that impact osteoclastogensis, and further that this marker could be assessed prior to or instead of determining therapeutic outcomes with standard joint histopathology techniques.

### Example 15

### Mice with Multiple Severely Swollen Paws have Elevated Serum OPG Levels

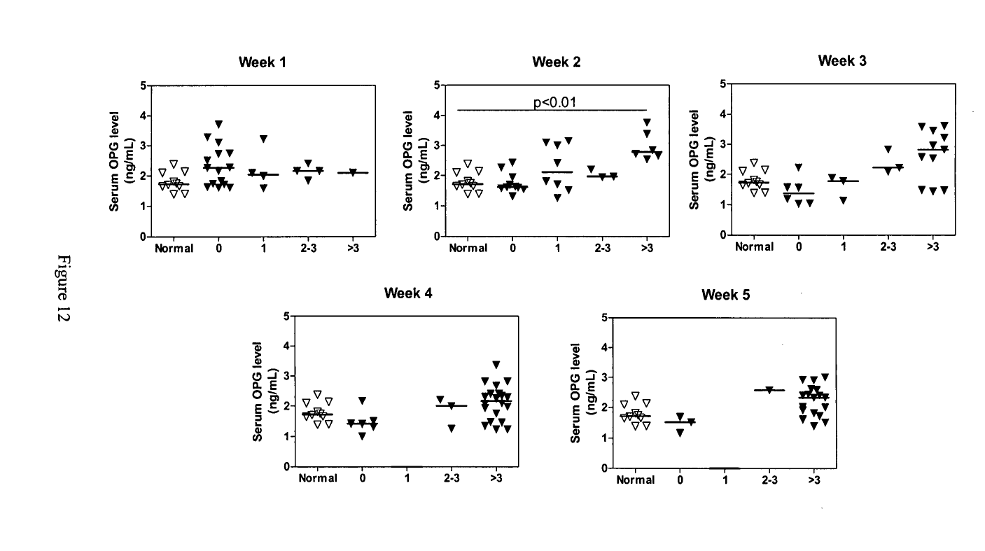

To assess the time course of the correlation between serum OPG levels and paw swelling, the experiment described in Example 14 was repeated using either a rat IgG1 isotype control antibody or a mouse IgG1 isotype control antibody and measuring the serum OPG levels instead of RANKL antibodies. The results with the rat IgG1 isotype control are shown in FIG. 12.

A proportion of not-yet-arthritic mice (DSS=0) at week 1 had elevated serum OPG levels that were still present due to the OPG elevation from the induction phase of the model. In contrast to the data generated in Example 13, where the average serum OPG levels in the “cohort of immunized and boosted” mice had fallen into the physiologic range from week 2 onward (FIG. 10, right panel), FIG. 12 shows a different picture. Serum OPG levels were “re-elevated” in the few mice with multiple severely swollen paws at week 2 over the more numerous other members of the cohort that were not-yet-arthritic. Mice with multiple severely swollen paws at later time points did not show greatly elevated serum OPG.

A similar weekly profile of serum OPG levels and mouse DSS was obtained when mice were dosed with mouse IgG1 isotype control (data not shown). In particular, elevated serum OPG levels were only seen in mice with multiple severely swollen paws with the level at week 2 being statistically significant and trends toward significance seen at weeks 3-4.

The above-described association between severe paw swelling and elevated serum OPG was only observed when the cohort of immunized and boosted mice were analyzed separately based on the disease severity. This was due to OPG's biphasic response in the CIA model wherein it was elevated in the induction phase and drops throughout the effector phase overlaid on a second profile of being re-elevated in mice with multiple severely swollen paws.

### Example 16

### JL7.1D10 Reduces Arthritis-associated Serum RANKL Levels

The results discussed in the above examples show (1) that elevated serum RANKL and OPG were seen in mice that had progressed to having at least one severely swollen paw and (2) that IL-17A neutralization inhibited bone erosion, even in the few severely swollen paws that were observed.

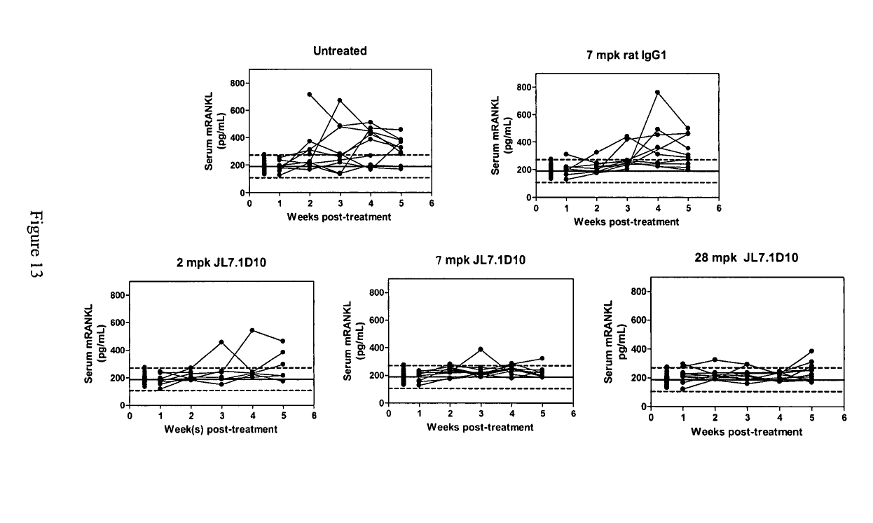

To assess whether anti-IL-17A neutralization modulates the severe arthritis-associated elevation in RANKL levels, mice were immunized, challenged, randomized to treatment at the first sign of severe paw swelling, and then treated subcutaneously weekly for 5 weeks with 7 mg/kg of rat IgG1 isotype control (25D2) mAb, or 2, 7, or 28 mg/kg of JL7.1D10 mAb. Serum drug concentrations were quantified over the course of the experiment to confirm drug exposure throughout the experiment. The serum RANKL levels were plotted over time from individual mice and the results are shown in FIG. 13.

Serum RANKL levels were elevated in untreated and isotype control mice (FIG. 13, upper left and right) over the time course of the CIA model, as also shown in FIG. 10 (left panel). Dosing with ≧7 mg/kg JL7.1D10 weekly for five weeks attenuated the arthritis-associated elevated serum RANKL levels (FIG. 13, lower middle panel). These results indicate that endogenous IL-17A plays a major role in RANKL production in CIA mice and that JL7.1D10 inhibition of RANKL expression may be partially the explanation for why JL7.1D10 inhibited joint bone erosion, even in the few paws that became severely swollen.

Further analysis of the data presented in FIGS. 11 and 13 showed that there were many fewer severely arthritis mice and many more non-arthritic mice in the JL7.1D10 treated group, and that serum RANKL levels were diminished even in the few JL7.1D10-treated mice that did progress to having multiple severely swollen paws compared to clinically equal control treated mice (data not shown). This result was statistically significant at week 4 and non-significant trends were observed at week 2, 3, and 5.

### Example 17

### JL7.1D10 Normalizes Serum OPG Levels Early in the Disease Progression Stage

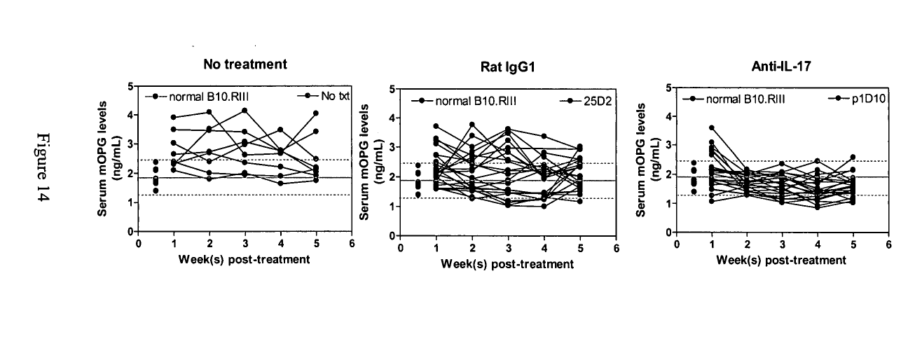

As discussed above, regulation of serum OPG levels is very complex in the mouse CIA model as evidenced by a biphasic component of OPG expression (i.e. elevation in the induction phase and a slow decrease into the disease progression phase) that is superimposed on a multiple-severely-swollen-paw-associated increase in serum OPG in the disease progression phase. To assess whether IL-17A neutralization within this complex regulation demonstrated any convincing modulation, mice were immunized, challenged, randomized to treatment at the first sign of severe paw swelling, and then treated subcutaneously weekly for 5 weeks with 7 mg/kg rat IgG1 isotype control (25D2) mAb or 28 mg/kg JL7.1D10 mAb. Serum drug concentrations were quantified over the course of the experiment to confirm drug exposure throughout the experiment. The weekly serum OPG profiles from individual mice are presented in FIG. 14.

Serum OPG concentrations were still elevated in both untreated and isotype control mice at the first time point studied in the disease progression phase and the average OPG level in the cohort of mice decreased over time, but with a very heterogeneous profile within the cohort. Weekly injections of 28 mg/kg JL7.1D10 for five weeks strikingly reduced serum OPG levels to near uniform levels within the physiologic range by the second week post-boost (FIG. 14, right panel).

To assess how IL-17A neutralization affected “re-elevated” OPG levels in the few “multiple-severely-swollen-paw” mice that broke through anti-IL-17A therapy, further analysis of the data presented in FIGS. 12 and 14 was conducted to measure the effect of IL-17A neutralization on the correlation between serum OPG concentrations versus “multiple-severely-swollen-paw” mice. JL7.1D10 reduced serum OPG levels in mice with multiple severely swollen paws (DSS>3) statistically at week 5 and with trends at earlier time points (data not shown).

### Example 18

### Effect of IL-17A on Serum RANKL and OPG Levels in Non-diseased Mice

The data discussed above indicate that IL-17A plays a significant role in RANKL and OPG production in arthritic mice and that IL-17A neutralization inhibits arthritis-associated bone erosion in mice presumably by inhibiting osteoclast differentiation/activity. To assess whether endogenous IL-17A had a significant role in normal bone homeostasis, serum RANKL or OPG levels were measured in normal B10.RIII mice before and during five weekly treatments of 28 mg/kg JL7.1D10 subcutaneously. The 1D10 treatment did not alter the physiologic serum RANKL or OPG levels in these normal mice, a result consistent with a conclusion that endogenous IL-17A is not critical for physiologic, non-inflammatory RANKL- and OPG-mediated bone biology in normal mice.

### Example 19

### Anti-IL-17A Treatment is Efficacious in Rat Adjuvant-induced Arthritis

The rat adjuvant-induced arthritis (AIA) is another example of a severe bone erosive model. The model is initiated by a single injection of Complete Fruend's Adjuvant (CFA) at the tail base, symmetric joint swelling responses start at day 10 to day 13, and severe bone erosion is seen by day 21.

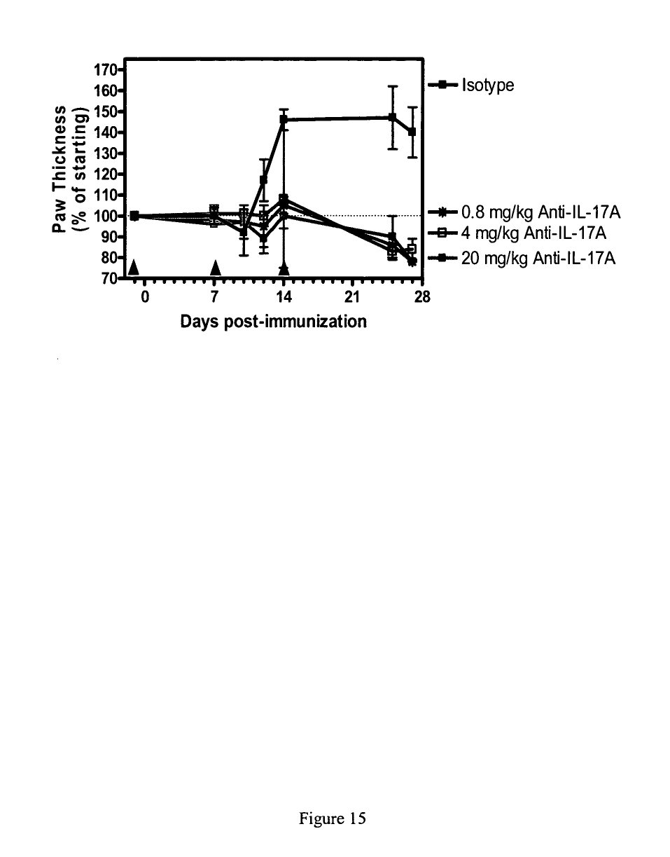

To investigate the role of IL-17A in the AIA model, an antibody that binds and neutralizes rat IL-17A was identified (JL8.18E10). Starting prior to CFA injection, dark Agouti rats were treated with weekly doses of an isotype antibody or 0.8, 4 or 20 mg/kg of JL8.18E10. The data, which are shown in FIG. 15, indicate that each dose of JL8.18E10 totally prevented arthritis onset. Similar experiments using the rat AIA model established that JL8.18E10 inhibited joint swelling whether administered at day 10 (disease onset) or day 12 (established disease) and also could inhibit the severe weight loss that is a property of AIA rats (data not shown).

To investigate whether IL-17A neutralization affects RANKL levels in the rat AIA model, RANKL was measured in serum harvested at sacrifice from rats dosed preventatively or at disease onset with JL8.18E10 or an isotype control. JL8.18E10 decreased serum RANKL in both treatment modes compared to isotype treated rats (data not shown).

### Example 20

### Anti-IL-17A Treatment Reduces Arthritis-associated Elevated Serum RANKL in Rat Adjuvant-induced Arthritis

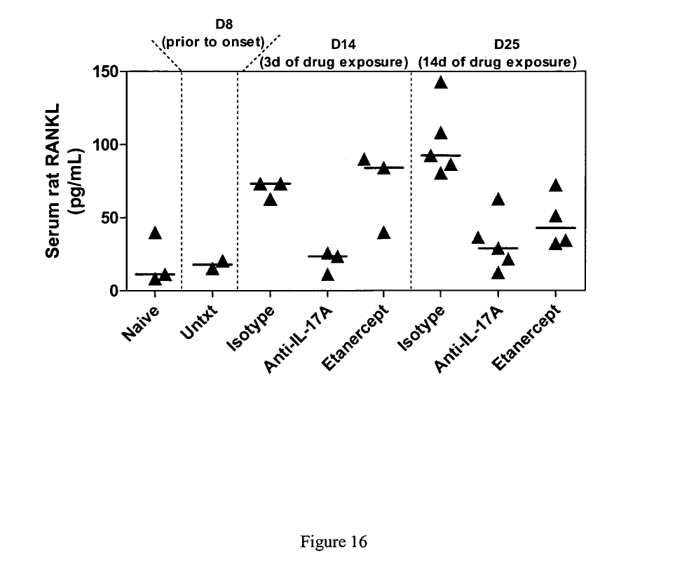

To assess the effect of IL-17A neutralization on RANKL in the AIA model, rats with established disease were treated with 20 mg/kg of an isotype control, a single 4 mg/kg or 20 mg/kg dose of JL8.18E10, or a 25 mg/kg dose of a TNF antagonist (etanercept) given every 3 days. The rats were bled at day 8 (prior to joint swelling), day 14 (three days after antibody treatment), and at the day 25 sacrifice. Serum RANKL was measured at each time point and the results are shown in FIG. 16.

Only three days of JL8.18E10 treatment was needed to inhibit the arthritis-associated elevated serum RANKL, while three days of TNF antagonism was not as effective in modulating serum RANKL. It should be noted that, at Day 14, the JL8.18E10 rats were still arthritic but their serum RANKL levels had been normalized.

Citation of the above publications or documents is not intended as an admission that any of the foregoing is pertinent prior art, nor does it constitute any admission as to the contents or date of these publications or documents. All references (publications, accession numbers, patent applications and patents) cited above are expressly incorporated by reference to the same extent as if each individual publication, accession number, patent application, or patent, was specifically and individually indicated to be incorporated by reference.

1-56. (canceled)

57. A method of selecting a patient with an inflammatory joint disease for treatment with an IL-17A antagonist, comprising
a. comparing the level of at least one joint destruction biomarker in a serum sample taken from the subject with the normal range of serum levels for the biomarker; and
b. selecting the patient for treatment with the IL-17A antagonist if the level of the joint destruction biomarker in the subject's serum sample is outside of the normal range,
wherein the inflammatory joint disease is rheumatoid arthritis, psoriatic arthritis or ankylosing spondylitis,
wherein the IL-17A antagonist is a monoclonal antibody or monoclonal antibody fragment that binds to and inhibits the activity of human IL-17A, and
wherein the joint destruction biomarker is selected from the group consisting of cartilage oligomer matrix protein (COMP), C-terminal cross-linking telopeptide of type I collagen (CTX-I), C-terminal cross-linking telopeptide of type II collagen (CTX-II), human cartilage glycoprotein-39 (HC gp-39), osteoprotegrin (OPG), Receptor activator of NFκB ligand (RANKL), osteocalcin and Tartrate-resistant acid phosphatase (TRACP) isoform 5b.

58. The method of claim 57, wherein the patient is an inflammatory non-responder to previous treatment with a different anti-rheumatic drug.

59. The method of claim 57, wherein the patient is an inflammatory responder to previous treatment with a different anti-rheumatic drug.

60. The method of claim 57, wherein the joint destruction biomarker is RANKL, COMP or OPG.

61. The method of claim 57, wherein the joint destruction biomarker is RANKL.

62. The method of claim 57, wherein the comparing step is performed on each of RANKL, COMP and OPG.

63. The method of claim 57, wherein the inflammatory joint disease is rheumatoid arthritis and the IL-17A antagonist is a humanized monoclonal antibody which comprises a light chain having SEQ ID NO:1 and a heavy chain having SEQ ID NO:2.

64. A method of predicting efficacy of an IL-17A antagonist in inhibiting joint destruction in a subject with an inflammatory joint disease, comprising:
a. determining the level of at least one joint destruction biomarker in a first serum sample taken from the subject prior to an initial treatment period with the IL-17A antagonist;
b. determining the level of the joint destruction biomarker in at least a second serum sample taken from the patient at the end of the initial treatment period; and
c. comparing the levels of the joint destruction biomarker in the first and second serum samples;
wherein a normalization of the level of the joint destruction biomarker in the second serum sample compared to the level in the first serum sample predicts that the IL-17A antagonist will likely be effective in inhibiting joint destruction in the subject,
wherein the inflammatory joint disease is rheumatoid arthritis, psoriatic arthritis or ankylosing spondylitis,
wherein the IL-17A antagonist is a monoclonal antibody or monoclonal antibody fragment that binds to and inhibits the activity of human IL-17A,
wherein the joint destruction biomarker is selected from the group consisting of cartilage oligomer matrix protein (COMP). C-terminal cross-linking telopeptide of type I collagen (CTX-I), C-terminal cross-linking telopeptide of type H collagen (CTX-II), human cartilage glycoprotein-39 (HC gp-39), osteoprotegrin (OPG), Receptor activator of NFκB ligand (RANKL), osteocalcin and Tartrate-resistant acid phosphatase (TRACP) isoform 5b, and wherein the subject is a human or a non-human animal.

65. The method of claim 64, wherein the initial treatment period is at least one week, at least two weeks, at least four weeks, at least eight weeks, at least twelve weeks, at least eighteen weeks, at least twenty-four weeks or at least forty-eight weeks.

66. The method of claim 64, further comprising comparing the level of the biomarker in the first and second serum samples with the normal range of serum levels of the biomarker, wherein the IL-17A antagonist is predicted to be effective in inhibiting joint destruction in the subject if the level of the joint destruction biomarker in the first serum sample is outside of the normal range and the level of the biomarker in the second serum sample falls within the normal range.

67. The method of claim 64, further comprising determining the level of the joint destruction biomarker in a third serum sample taken from the subject at the end of at least one subsequent treatment period with the IL-17A antagonist, wherein a level of the biomarker in the third serum sample that is more normalized than the level of the biomarker in the second serum sample predicts that the IL-17A antagonist will likely be effective in inhibiting joint destruction in the subject.

68. The method of claim 67, wherein the subsequent treatment period is at least 12 weeks, at least 24 weeks or at least 48 weeks.

69. The method of claim 64, wherein the biomarker is RANKL, COMP or OPG.

70. The method of claim 64, wherein the biomarker is RANKL.

71. The method of claim 64, wherein the determining and comparing steps are performed on each of RANKL and COMP or on each of RANKL, COMP and OPG.

72. The method of claim 64, wherein the subject is a human and the IL-17A antagonist is a humanized monoclonal antibody, a humanized monoclonal antibody fragment, a fully human monoclonal antibody or a fully human monoclonal antibody fragment.

73. The method of claim 72, wherein the IL-17A antagonist is a humanized monoclonal antibody or a fully human monoclonal antibody and the subject is treated during the initial treatment period with a dose of the antibody that has been shown to be effective in inhibiting joint destruction in a population of subjects with the inflammatory joint disease.

74. The method of claim 72, wherein the inflammatory joint disease is rheumatoid arthritis and the IL-17A antagonist is a humanized monoclonal antibody which comprises a light chain having SEQ ID NO:1 and a heavy chain having SEQ ID NO:2.

75. The method of claim 72, wherein the subject is an inflammatory non-responder to previous treatment with a different anti-rheumatic drug.

76. The method of claim 72, wherein the subject is an inflammatory responder to previous treatment with a different anti-rheumatic drug.

77. A method of treating a human subject for an inflammatory joint disease with an IL-17A antagonist, comprising
a. determining the level of at least one joint destruction biomarker in a first serum sample taken from the subject;
b. administering the IL-17A antagonist to the subject according to a first dosing regimen during an initial treatment period;
c. determining the level of the joint destruction biomarker in at least a second serum sample taken from the patient at the end of the initial treatment period; and
d. comparing the levels of the biomarker in the first and second serum samples; and
e. administering the IL-17A antagonist to the subject according to the first dosing regimen during at least one subsequent treatment period if the level of the biomarker in the second serum sample is within a specified range; or
f. administering the IL-17A antagonist to the subject according to a second dosing regimen during at least one subsequent treatment period if the level of the biomarker in the second serum sample is outside of the specified range, wherein the second dosing regimen comprises administering a total amount of the IL-17A antagonist during the subsequent treatment period that is higher than the total amount administered during the initial treatment period,
wherein the specified range is selected from the group consisting of:
(i) the range of serum levels of the joint destruction biomarker found in untreated subjects who do not have the inflammatory joint disease; and
(ii) the range defined by a confidence interval of at least 80% of the mean level of the joint destruction biomarker measured in a population of subjects with the inflammatory joint disease who were treated with the IL-17A antagonist according to the first dosing regimen for a time period equal to or longer than the initial treatment period, wherein the population exhibited inhibition of joint destruction following treatment with the IL-17A antagonist according to the first dosing regimen during a time period equal to or longer than the subsequent treatment period,
wherein the inflammatory joint disease is rheumatoid arthritis, psoriatic arthritis or ankylosing spondylitis,
wherein the IL-17A antagonist is a monoclonal antibody or monoclonal antibody fragment that binds to and inhibits the activity of human IL-17A, and
wherein the joint destruction biomarker is selected from the group consisting of cartilage oligomer matrix protein (COMP), C-terminal cross-linking telopeptide of type I collagen (CTX-I), C-terminal cross-linking telopeptide of type II collagen (CTX-II), human cartilage glycoprotein-39 (HC gp-39), osteoprotegrin (OPG), Receptor activator of NFκB ligand (RANKL), osteocalcin and Tartrate-resistant acid phosphatase (TRACP) isoform 5b.

78. The method of claim 77, wherein the specified range is defined by a confidence interval of at least 85%, at least 90% or at least 95% of the mean level of the joint destruction biomarker measured in a population of subjects with the inflammatory disease who were treated with the IL-17A antagonist according to the first dosing regimen for an initial time period of at least 4 weeks, wherein the population exhibited inhibition of joint destruction following treatment with the IL-17A antagonist according to the first dosing regimen during a subsequent treatment period of at least 12 weeks.

79. The method of claim 77, wherein the initial treatment period is at least one week, at least two weeks, at least four weeks, at least eight weeks or at least twelve weeks.

80. The method of claim 77, wherein the subsequent treatment period is at least 12 weeks, at least 24 weeks or at least 48 weeks.

81. The method of claim 77, wherein the inflammatory joint disease is rheumatoid arthritis, the IL-17A antagonist is a humanized monoclonal antibody which comprises a light chain having SEQ ID NO:1 and a heavy chain having SEQ ID NO:2 and the joint destruction biomarker is RANKL.

82. The method of claim 77, wherein the subject is an inflammatory non-responder to previous treatment with a different anti-rheumatic drug.

83. The method of claim 77, wherein the subject is an inflammatory responder to previous treatment with a different anti-rheumatic drug.

84. The method of claim 77, wherein the IL-17 antagonist is an antibody that does not bind to IL-23 and the method further comprises administering an IL-23 antagonist to the patient during the initial treatment period, during the subsequent treatment period, or during both the initial and subsequent treatment periods.

85. A kit for treating an inflammatory joint disease, wherein the kit comprises a pharmaceutical composition and reagents for measuring the level of at least one joint destruction biomarker in a serum sample taken from a subject, wherein the pharmaceutical composition comprises an IL-17A antagonist and the joint destruction biomarker is selected from the group consisting of cartilage oligomer matrix protein (COMP), C-terminal cross-linking telopeptide of type I collagen (CTX-I), C-terminal cross-linking telopeptide of type II collagen (CTX-II), human cartilage glycoprotein-39 (HC gp-39), osteoprotegrin (OPG), Receptor activator of NFκB ligand (RANKL), osteocalcin and Tartrate-resistant acid phosphatase (TRACP) isoform 5b.

86. The kit of claim 85, wherein the inflammatory joint disease is rheumatoid arthritis, the IL-17A antagonist is a humanized monoclonal antibody which comprises a light chain having SEQ ID NO:1 and a heavy chain having SEQ ID NO:2, and the joint destruction biomarker is RANKL.

87. A manufactured drug product for treating an inflammatory joint disease, which comprises a pharmaceutical formulation comprising an IL-17A antagonist and instructions for determining patient serum levels of at least one joint destruction biomarker before and during treatment with the IL-17A antagonist,
wherein the inflammatory joint disease is rheumatoid arthritis, the IL-17A antagonist is a humanized monoclonal antibody which comprises a light chain having SEQ ID NO:1 and a heavy chain having SEQ ID NO:2, and the joint destruction biomarker is RANKL, COMP or OPG.

88. The manufactured drug product of claim 87, wherein the instructions further comprise recommending use of the pharmaceutical formulation for treating patients who have an abnormal level of the biomarker following previous therapy with a different anti-rheumatic drug.

89. The manufactured drug product of claim 87, wherein the instructions further comprise recommending use of the pharmaceutical formulation for treating patients who are inflammatory non-responders following previous therapy with a different anti-rheumatic drug.
